SEQUENCE LISTING (18 sequences)

SEQ ID NO: 1 - protein, 540 aa, Norovirus
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNDYDPTEEIPAPLGTPDFVGKIQGVLTQTTRTDGSTRGHKATVYTGSADFAPKLGRVQFETDTDRDFEANQNTKFTPVGVIQDGGTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 2 - protein, 268 aa, Norovirus
MAGAFFAGLASDVLGSGLGSLINAGAGAINQKVEFENNRKLQQASFQFSSNLQQASFQHDKEMLQAQIEATKKLQQEMMKVKQAMLLEGGFFETDAARGAINAPMTKALDWSGTRYWAPDARTTTYNAGRFSTPQPSGALPGRANLRDAVPARGSSSKSSNSSTATSVYSNQTTSTRLGSTAVSGTSVSSFPSTARTRSWVEDQSRNLSPSMRGAHNISFVTPPSSRSSSQGTVSTVPKEVLDSWTGAFNTRRQPLFAHIRKRGESRA

SEQ ID NO: 3 - DNA, 1623 bp, Norovirus
atgaagatggcgtcgagtgacgccaacccatctgatgggtccgcagccaacctcgtcccagaggtcaacaatgaggttatggctctggagcccgttgttggtgccgccattgcggcacctgtagcgggccaacaaaatgtaattgacccctggattagaaataattttgtacaagcccctggtggagagtttacagtatcccctagaaacgctccaggtgaaatactatggagcgcgcccttgggccctgatctaaatccctacctatcccatttggccagaatgtacaatggttatgcaggtggttttgaagtgcaggtaattctcgcggggaacgcgttcaccgccgggaaggtcatatttgcagcagtcccaccaaattttccaactgaaggcttgagccccagccaggtcactatgttcccccatatagtagtagatgttaggcaactagaacctgtgttgattcccttacccgatgttaggaataatttctatcattataatcaatcaaatgaccctaccattaagttgatagcaatgttgtatacaccacttagggctaataatgctggggatgatgtcttcacagtttcttgccgagttctcacgagaccatcccccgattttgatttcatatttttagtgccacccacagttgagtcaagaactaaaccattctctgtcccagttttaactgttgaggagatgaccaattcaagattccccattcctttggaaaagttgttcacgggtcccagcagtgcctttgttgtccaaccacaaaacggcaggtgcacgactgatggcgtgctcctaggcaccacccaactgtctcctgtcaacatctgcaccttcagaggagatgtcacccacatcacaggtagtcgtaactacacaatgaatttggcttctcaaaattggaacgattatgacccaacagaagaaatcccagcccctctaggaactccagattttgtggggaagattcaaggcgttctcacccaaaccacaaggacagatggctcaacacgcggccacaaagctacagtgtacactgggagcgccgactttgctccaaaactgggtagagttcaatttgaaactgacacagaccgtgattttgaagctaaccaaaacacaaagttcaccccagtcggtgtcatccaagatggtggcaccacccaccgaaatgaaccccaacagtgggtgctcccaagctactcaggcagaaatactcataatgtgcatctggcccccgctgtagcccccacttttccgggtgagcaacttctcttcttcagatccaccatgcccggatgcagcgggtaccccaacatggatttggactgtctgctcccccaggaatgggtgcagtacttctaccaagaggcagccccagcacaatctgatgtggctctgctaagatttgtgaatccagacacaggtagggttttgtttgagtgtaagcttcataaatcaggctatgttacagtggctcacactggccaacatgatttggttatcccccccaatggttattttaggtttgattcctgggtcaaccagttttacacgcttgcccccatgggaaatggaacggggcgtagacgtgcagtataa

SEQ ID NO: 4 - DNA, 804 bp, Norovirus
atggctggagctttctttgctggattggcatctgatgtccttggctctgggcttggttcccttatcaatgctggggctggggccatcaaccaaaaagttgagtttgaaaataacagaaaattgcaacaagcatccttccaatttagcagcaatctacaacaggcttcctttcaacatgacaaagagatgctccaagcacaaattgaggccaccaaaaagctacaacaggaaatgatgaaagttaagcaggcaatgctcctagagggtgggttctttgagacagatgcagcccgcggggcaatcaacgcccccatgacaaaagctttggactggagcgggacaaggtactgggctcccgatgctaggactacaacatacaatgcaggccgcttttccacccctcaaccatcgggggcactgccaggaagagctaatcttagggatgctgtccctgctcggggttcctccagcaaatcttctaactcttctactgctacttctgtgtactcaaatcaaactacttcaacgagacttggttctacagctgtttctggtaccagtgtctcgagcttcccgtcaactgcaaggactaggagctgggttgaggatcaaagtaggaatttgtcaccttccatgaggggggcccacaacatatcgtttgtcaccccaccatctagcagatcctctagccaaggcacagtctcaaccgtgcctaaagaggttttggactcctggactggcgctttcaacacgcgcaggcagccactcttcgctcacattcgtaagcgaggggagtcacgggcg

SEQ ID NO: 5 - DNA, 1623 bp, Artificial Sequence
  Codon optimized VP1
atgaagatggcttcctctgacgccaatccttccgatggaagcgctgctaatctggtgcccgaggtcaacaacgaggtaatggcactggaacccgtcgtgggtgcagcgattgccgcccctgttgctggacagcagaatgtgatcgatccctggatacgcaataatttcgtgcaggcgcctggaggcgaattcacggtgtctcctagaaatgccccaggcgaaattctgtggtcagctcctctggggcccgacttgaacccatacctcagtcatctcgcacgaatgtacaacgggtatgccggaggctttgaagtgcaggtgatccttgccggaaacgcattcaccgccgggaaagtcatcttcgccgctgtcccacccaatttccctaccgaagggctttcaccaagtcaggtaaccatgtttccacacatcgttgtggatgtccgccagcttgaacctgtactcatccctctgccagatgttaggaacaacttctaccactacaaccagagcaacgatccaaccattaagctgatcgccatgttgtatacaccactcagagccaataacgcaggcgatgatgtgttcactgtctcttgtagagtcctgacacgaccctctcccgatttcgacttcattttcctcgtgccacccaccgttgagagccggacaaaaccgtttagcgtacccgtgctgactgtcgaggagatgaccaattctaggttccccattcctctggagaagctgtttactggaccgtccagcgcctttgttgtgcagccacagaacggccgctgtactactgatggcgtgctgcttggtacaacccaactgagcccagtgaatatctgcaccttcagaggggacgtgactcatatcaccggatcccgcaactacacaatgaacctggcttcacagaactggaatgactacgatccaacggaggaaataccggctcctctgggtactccggacttcgtcggcaagattcagggagtgctgacccaaactacccgtactgatgggagcacccgtggccacaaagctaccgtgtatacaggcagtgctgactttgcgcctaaactgggtagggtgcaatttgagactgacaccgatcgggactttgaggccaatcagaataccaagtttacacccgtcggcgtcatacaggacggcgggacgacacatcgcaacgaaccccagcaatgggtattgccctcctattccggtagaaacacgcacaacgttcacttggcaccagccgtggcccctacctttcctggagagcaactgctgttcttccgatctacaatgccggggtgtagtggctatccaaacatggacctcgactgcctcctgccccaagagtgggttcagtacttttatcaggaagccgcacccgcccagagcgacgttgcactgttgcggtttgtaaatcccgatacgggtcgggtgctcttcgagtgcaagctgcataaatcaggctatgtcacagtggcacacacagggcaacatgaccttgtgattccgcctaacggttacttccggtttgacagctgggtgaatcagttttacactctggctcctatgggaaatgggacaggcaggagaagggcggtttga

SEQ ID NO: 6 - DNA, 804 bp, Artificial Sequence
  Codon optimized VP2
atggcaggagccttctttgcaggattggcaagcgatgttctgggctccggacttggttccctgatcaacgcaggagctggcgccatcaatcagaaggtcgaattcgagaacaatcggaagctgcagcaagcttcctttcagttctctagcaatctgcagcaagcctcatttcagcatgacaaagagatgctgcaggctcagatagaggccacgaagaaactgcagcaagagatgatgaaagtgaagcaagcgatgctccttgaaggtggcttctttgagactgacgctgcgagaggcgccattaatgccccgatgacgaaagccctggactggtcagggaccaggtattgggctcctgatgcccgcaccactacatacaacgccggtcgcttttccactcctcaaccatcaggggcactcccaggacgggcgaatctgagggatgctgtgcccgcaagaggcagcagctccaagagcagtaacagctcaaccgctacaagcgtgtacagcaaccagacgacctcaacccggttgggctccacagccgtatctggtacctctgtgagctctttcccctctactgcccgtactcgaagttgggtggaagatcagtcccggaatctctctcccagtatgcgtggggctcacaacatcagcttcgttaccccaccctctagtcgctcaagtagccagggcacagtgtccacagtccctaaagaagtcctggactcctggacaggggcctttaacaccagaaggcagcctctcttcgcccacattaggaagagaggggagagtcgagca

SEQ ID NO: 7 - DNA, 2526 bp, Artificial Sequence
  VP1-linker-VP2 construct
cgtacggccaccatgaagatggcgtcgagtgacgccaacccatctgatgggtccgcagccaacctcgtcccagaggtcaacaatgaggttatggctctggagcccgttgttggtgccgccattgcggcacctgtagcgggccaacaaaatgtaattgacccctggattagaaataattttgtacaagcccctggtggagagtttacagtatcccctagaaacgctccaggtgaaatactatggagcgcgcccttgggccctgatctaaatccctacctatcccatttggccagaatgtacaatggttatgcaggtggttttgaagtgcaggtaattctcgcggggaacgcgttcaccgccgggaaggtcatatttgcagcagtcccaccaaattttccaactgaaggcttgagccccagccaggtcactatgttcccccatatagtagtagatgttaggcaactagaacctgtgttgattcccttacccgatgttaggaataatttctatcattataatcaatcaaatgaccctaccattaagttgatagcaatgttgtatacaccacttagggctaataatgctggggatgatgtcttcacagtttcttgccgagttctcacgagaccatcccccgattttgatttcatatttttagtgccacccacagttgagtcaagaactaaaccattctctgtcccagttttaactgttgaggagatgaccaattcaagattccccattcctttggaaaagttgttcacgggtcccagcagtgcctttgttgtccaaccacaaaacggcaggtgcacgactgatggcgtgctcctaggcaccacccaactgtctcctgtcaacatctgcaccttcagaggagatgtcacccacatcacaggtagtcgtaactacacaatgaatttggcttctcaaaattggaacgattatgacccaacagaagaaatcccagcccctctaggaactccagattttgtggggaagattcaaggcgttctcacccaaaccacaaggacagatggctcaacacgcggccacaaagctacagtgtacactgggagcgccgactttgctccaaaactgggtagagttcaatttgaaactgacacagaccgtgattttgaagctaaccaaaacacaaagttcaccccagtcggtgtcatccaagatggtggcaccacccaccgaaatgaaccccaacagtgggtgctcccaagctactcaggcagaaatactcataatgtgcatctggcccccgctgtagcccccacttttccgggtgagcaacttctcttcttcagatccaccatgcccggatgcagcgggtaccccaacatggatttggactgtctgctcccccaggaatgggtgcagtacttctaccaagaggcagccccagcacaatctgatgtggctctgctaagatttgtgaatccagacacaggtagggttttgtttgagtgtaagcttcataaatcaggctatgttacagtggctcacactggccaacatgatttggttatcccccccaatggttattttaggtttgattcctgggtcaaccagttttacacgcttgcccccatgggaaatggaacggggcgtagacgtgcagtataatagcctaccctccatcattgttataaaaaacttaggaaccaggtccacacagccgccagcccatcaacggccaccatggctggagctttctttgctggattggcatctgatgtccttggctctgggcttggttcccttatcaatgctggggctggggccatcaaccaaaaagttgagtttgaaaataacagaaaattgcaacaagcatccttccaatttagcagcaatctacaacaggcttcctttcaacatgacaaagagatgctccaagcacaaattgaggccaccaaaaagctacaacaggaaatgatgaaagttaagcaggcaatgctcctagagggtgggttctttgagacagatgcagcccgcggggcaatcaacgcccccatgacaaaagctttggactggagcgggacaaggtactgggctcccgatgctaggactacaacatacaatgcaggccgcttttccacccctcaaccatcgggggcactgccaggaagagctaatcttagggatgctgtccctgctcggggttcctccagcaaatcttctaactcttctactgctacttctgtgtactcaaatcaaactacttcaacgagacttggttctacagctgtttctggtaccagtgtctcgagcttcccgtcaactgcaaggactaggagctgggttgaggatcaaagtaggaatttgtcaccttccatgaggggggcccacaacatatcgtttgtcaccccaccatctagcagatcctctagccaaggcacagtctcaaccgtgcctaaagaggttttggactcctggactggcgctttcaacacgcgcaggcagccactcttcgctcacattcgtaagcgaggggagtcacgggcgtaatgagcgcgc

SEQ ID NO: 8 - DNA, 2526 bp, Artificial Sequence
  VP1-linker-VP2 construct codon optimized
cgtacggccaccatgaagatggcttcctctgacgccaatccttccgatggaagcgctgctaatctggtgcccgaggtcaacaacgaggtaatggcactggaacccgtcgtgggtgcagcgattgccgcccctgttgctggacagcagaatgtgatcgatccctggatacgcaataatttcgtgcaggcgcctggaggcgaattcacggtgtctcctagaaatgccccaggcgaaattctgtggtcagctcctctggggcccgacttgaacccatacctcagtcatctcgcacgaatgtacaacgggtatgccggaggctttgaagtgcaggtgatccttgccggaaacgcattcaccgccgggaaagtcatcttcgccgctgtcccacccaatttccctaccgaagggctttcaccaagtcaggtaaccatgtttccacacatcgttgtggatgtccgccagcttgaacctgtactcatccctctgccagatgttaggaacaacttctaccactacaaccagagcaacgatccaaccattaagctgatcgccatgttgtatacaccactcagagccaataacgcaggcgatgatgtgttcactgtctcttgtagagtcctgacacgaccctctcccgatttcgacttcattttcctcgtgccacccaccgttgagagccggacaaaaccgtttagcgtacccgtgctgactgtcgaggagatgaccaattctaggttccccattcctctggagaagctgtttactggaccgtccagcgcctttgttgtgcagccacagaacggccgctgtactactgatggcgtgctgcttggtacaacccaactgagcccagtgaatatctgcaccttcagaggggacgtgactcatatcaccggatcccgcaactacacaatgaacctggcttcacagaactggaatgactacgatccaacggaggaaataccggctcctctgggtactccggacttcgtcggcaagattcagggagtgctgacccaaactacccgtactgatgggagcacccgtggccacaaagctaccgtgtatacaggcagtgctgactttgcgcctaaactgggtagggtgcaatttgagactgacaccgatcgggactttgaggccaatcagaataccaagtttacacccgtcggcgtcatacaggacggcgggacgacacatcgcaacgaaccccagcaatgggtattgccctcctattccggtagaaacacgcacaacgttcacttggcaccagccgtggcccctacctttcctggagagcaactgctgttcttccgatctacaatgccggggtgtagtggctatccaaacatggacctcgactgcctcctgccccaagagtgggttcagtacttttatcaggaagccgcacccgcccagagcgacgttgcactgttgcggtttgtaaatcccgatacgggtcgggtgctcttcgagtgcaagctgcataaatcaggctatgtcacagtggcacacacagggcaacatgaccttgtgattccgcctaacggttacttccggtttgacagctgggtgaatcagttttacactctggctcctatgggaaatgggacaggcaggagaagggcggtttgatagcctaccctccatcattgttataaaaaacttaggaaccaggtccacacagccgccagcccatcaacggccaccatggcaggagccttctttgcaggattggcaagcgatgttctgggctccggacttggttccctgatcaacgcaggagctggcgccatcaatcagaaggtcgaattcgagaacaatcggaagctgcagcaagcttcctttcagttctctagcaatctgcagcaagcctcatttcagcatgacaaagagatgctgcaggctcagatagaggccacgaagaaactgcagcaagagatgatgaaagtgaagcaagcgatgctccttgaaggtggcttctttgagactgacgctgcgagaggcgccattaatgccccgatgacgaaagccctggactggtcagggaccaggtattgggctcctgatgcccgcaccactacatacaacgccggtcgcttttccactcctcaaccatcaggggcactcccaggacgggcgaatctgagggatgctgtgcccgcaagaggcagcagctccaagagcagtaacagctcaaccgctacaagcgtgtacagcaaccagacgacctcaacccggttgggctccacagccgtatctggtacctctgtgagctctttcccctctactgcccgtactcgaagttgggtggaagatcagtcccggaatctctctcccagtatgcgtggggctcacaacatcagcttcgttaccccaccctctagtcgctcaagtagccagggcacagtgtccacagtccctaaagaagtcctggactcctggacaggggcctttaacaccagaaggcagcctctcttcgcccacattaggaagagaggggagagtcgagcataatgagcgcgc

SEQ ID NO: 9 - DNA, 17634 bp, Artificial Sequence
  MV Noro VP1 optimized cDNA plus strand
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggccaccatgaagatggcctcttctgacgccaacccttcagacggctcagctgccaatttggtgcctgaggtgaacaacgaggtgatggccctggaacccgtggtcggagccgctatagccgcaccagttgccggccagcagaatgtcatcgacccgtggattcggaataacttcgtgcaagcgccgggcggtgagttcaccgtcagcccacgaaatgccccaggcgagatcctgtggagtgctccattggggcctgatcttaacccatacctgtcacacttggcgagaatgtacaatgggtatgcaggaggatttgaggtccaggttattctggctgggaatgctttcacagcagggaaggtgattttcgccgccgtccctcctaactttcccacagagggtctgagcccaagtcaggtcaccatgttcccacacattgtggtagatgtgagacagctcgagcctgtcctgattccacttccagacgttaggaacaacttttaccactacaatcagagcaacgatcccaccatcaagctcattgccatgctgtatacacccctgagagccaacaatgctggggatgacgtctttactgtgagctgtcgcgttctgacgcggccctcccctgactttgacttcatcttcctcgttcctccaacggtggaatccaggacgaagccattctcagtccctgtcctcactgtggaggaaatgaccaatagcaggtttcctatccctctggagaaactgttcactggcccgagctccgcatttgtggtacagcctcagaacgggagatgtaccacagatggcgtgcttctcggcacaacccaactctctccagtgaatatctgcacgtttcgtggcgacgtaacccacatcactggaagcaggaattacaccatgaatctcgccagtcaaaactggaacgactacgaccccacagaagaaatacccgcacccctggggacaccagattttgtgggtaaaatccagggagtactgactcaaaccacccgaactgacggttctactcggggccataaagccactgtgtataccgggagcgcggatttcgctcccaaattgggacgcgtacagtttgagacagatactgatcgggacttcgaagcaaaccagaacaccaagttcactcccgttggcgttattcaggatggaggcaccacccatcgaaatgagccgcagcaatgggtactgccctcctacagtggacggaatacgcataacgtgcatctggctcctgcagttgctcctactttccccggcgagcagctgctgttcttccgctctaccatgcccgggtgtagcgggtatcccaacatggaccttgactgcttgcttccccaagaatgggtgcagtacttctatcaggaagccgctcctgcgcagtctgatgtggcactcctgcgctttgtgaacccggatacaggaagagtgctgtttgaatgcaagctgcacaaatccggatacgtgacagtcgctcacacaggtcaacatgatctggttataccaccgaatggctatttccgctttgactcctgggtgaatcagttttataccctggcacccatgggtaacggtacaggccgtaggagagccgtctaatgagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 10 - DNA, 18516 bp, Artificial Sequence
  MV Noro VP1 ATU VP2 optimized cDNA plus strand
accaaacaaagttgggtaaggatagttcaatcaatgatcatcttctagtgcacttaggattcaagatcctattatcagggacaagagcaggattagggatatccgagatggccacacttttaaggagcttagcattgttcaaaagaaacaaggacaaaccacccattacatcaggatccggtggagccatcagaggaatcaaacacattattatagtaccaatccctggagattcctcaattaccactcgatccagacttctggaccggttggtgaggttaattggaaacccggatgtgagcgggcccaaactaacaggggcactaataggtatattatccttatttgtggagtctccaggtcaattgattcagaggatcaccgatgaccctgacgttagcataaggctgttagaggttgtccagagtgaccagtcacaatctggccttaccttcgcatcaagaggtaccaacatggaggatgaggcggaccaatacttttcacatgatgatccaattagtagtgatcaatccaggttcggatggttcgggaacaaggaaatctcagatattgaagtgcaagaccctgagggattcaacatgattctgggtaccatcctagcccaaatttgggtcttgctcgcaaaggcggttacggccccagacacggcagctgattcggagctaagaaggtggataaagtacacccaacaaagaagggtagttggtgaatttagattggagagaaaatggttggatgtggtgaggaacaggattgccgaggacctctccttacgccgattcatggtcgctctaatcctggatatcaagagaacacccggaaacaaacccaggattgctgaaatgatatgtgacattgatacatatatcgtagaggcaggattagccagttttatcctgactattaagtttgggatagaaactatgtatcctgctcttggactgcatgaatttgctggtgagttatccacacttgagtccttgatgaacctttaccagcaaatgggggaaactgcaccctacatggtaatcctggagaactcaattcagaacaagttcagtgcaggatcataccctctgctctggagctatgccatgggagtaggagtggaacttgaaaactccatgggaggtttgaactttggccgatcttactttgatccagcatattttagattagggcaagagatggtaaggaggtcagctggaaaggtcagttccacattggcatctgaactcggtatcactgccgaggatgcaaggcttgtttcagagattgcaatgcatactactgaggacaagatcagtagagcggttggacccagacaagcccaagtatcatttctacacggtgatcaaagtgagaatgagctaccgagattggggggcaaggaagataggagggtcaaacagagtcgaggagaagccagggagagctacagagaaaccgggcccagcagagcaagtgatgcgagagctgcccatcttccaaccggcacacccctagacattgacactgcaacggagtccagccaagatccgcaggacagtcgaaggtcagctgacgccctgcttaggctgcaagccatggcaggaatctcggaagaacaaggctcagacacggacacccctatagtgtacaatgacagaaatcttctagactaggtgcgagaggccgagggccagaacaacatccgcctaccatccatcattgttataaaaaacttaggaaccaggtccacacagccgccagcccatcaaccatccactcccacgattggagccaatggcagaagagcaggcacgccatgtcaaaaacggactggaatgcatccgggctctcaaggccgagcccatcggctcactggccatcgaggaagctatggcagcatggtcagaaatatcagacaacccaggacaggagcgagccacctgcagggaagagaaggcaggcagttcgggtctcagcaaaccatgcctctcagcaattggatcaactgaaggcggtgcacctcgcatccgcggtcagggacctggagagagcgatgacgacgctgaaactttgggaatccccccaagaaatctccaggcatcaagcactgggttacagtgttattacgtttatgatcacagcggtgaagcggttaagggaatccaagatgctgactctatcatggttcaatcaggccttgatggtgatagcaccctctcaggaggagacaatgaatctgaaaacagcgatgtggatattggcgaacctgataccgagggatatgctatcactgaccggggatctgctcccatctctatggggttcagggcttctgatgttgaaactgcagaaggaggggagatccacgagctcctgagactccaatccagaggcaacaactttccgaagcttgggaaaactctcaatgttcctccgcccccggaccccggtagggccagcacttccgggacacccattaaaaagggcacagacgcgagattagcctcatttggaacggagatcgcgtctttattgacaggtggtgcaacccaatgtgctcgaaagtcaccctcggaaccatcagggccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctctgatgtccaagatattaaaacagccttggccaaaatacacgaggataatcagaagataatctccaagctagaatcactgctgttattgaagggagaagttgagtcaattaagaagcagatcaacaggcaaaatatcagcatatccaccctggaaggacacctctcaagcatcatgatcgccattcctggacttgggaaggatcccaacgaccccactgcagatgtcgaaatcaatcccgacttgaaacccatcataggcagagattcaggccgagcactggccgaagttctcaagaaacccgttgccagccgacaactccaaggaatgacaaatggacggaccagttccagaggacagctgctgaaggaatttcagctaaagccgatcgggaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaatgatcttgccaagttccaccagatgctgatgaagataataatgaagtagctacagctcaacttacctgccaaccccatgccagtcgacccaactagcctaccctccatcattgttataaaaaacttaggaaccaggtccacacagccgccagcccatcaacgcgtacggccaccatgaagatggcttcctctgacgccaatccttccgatggaagcgctgctaatctggtgcccgaggtcaacaacgaggtaatggcactggaacccgtcgtgggtgcagcgattgccgcccctgttgctggacagcagaatgtgatcgatccctggatacgcaataatttcgtgcaggcgcctggaggcgaattcacggtgtctcctagaaatgccccaggcgaaattctgtggtcagctcctctggggcccgacttgaacccatacctcagtcatctcgcacgaatgtacaacgggtatgccggaggctttgaagtgcaggtgatccttgccggaaacgcattcaccgccgggaaagtcatcttcgccgctgtcccacccaatttccctaccgaagggctttcaccaagtcaggtaaccatgtttccacacatcgttgtggatgtccgccagcttgaacctgtactcatccctctgccagatgttaggaacaacttctaccactacaaccagagcaacgatccaaccattaagctgatcgccatgttgtatacaccactcagagccaataacgcaggcgatgatgtgttcactgtctcttgtagagtcctgacacgaccctctcccgatttcgacttcattttcctcgtgccacccaccgttgagagccggacaaaaccgtttagcgtacccgtgctgactgtcgaggagatgaccaattctaggttccccattcctctggagaagctgtttactggaccgtccagcgcctttgttgtgcagccacagaacggccgctgtactactgatggcgtgctgcttggtacaacccaactgagcccagtgaatatctgcaccttcagaggggacgtgactcatatcaccggatcccgcaactacacaatgaacctggcttcacagaactggaatgactacgatccaacggaggaaataccggctcctctgggtactccggacttcgtcggcaagattcagggagtgctgacccaaactacccgtactgatgggagcacccgtggccacaaagctaccgtgtatacaggcagtgctgactttgcgcctaaactgggtagggtgcaatttgagactgacaccgatcgggactttgaggccaatcagaataccaagtttacacccgtcggcgtcatacaggacggcgggacgacacatcgcaacgaaccccagcaatgggtattgccctcctattccggtagaaacacgcacaacgttcacttggcaccagccgtggcccctacctttcctggagagcaactgctgttcttccgatctacaatgccggggtgtagtggctatccaaacatggacctcgactgcctcctgccccaagagtgggttcagtacttttatcaggaagccgcacccgcccagagcgacgttgcactgttgcggtttgtaaatcccgatacgggtcgggtgctcttcgagtgcaagctgcataaatcaggctatgtcacagtggcacacacagggcaacatgaccttgtgattccgcctaacggttacttccggtttgacagctgggtgaatcagttttacactctggctcctatgggaaatgggacaggcaggagaagggcggtttgatagcctaccctccatcattgttataaaaaacttaggaaccaggtccacacagccgccagcccatcaacggccaccatggcaggagccttctttgcaggattggcaagcgatgttctgggctccggacttggttccctgatcaacgcaggagctggcgccatcaatcagaaggtcgaattcgagaacaatcggaagctgcagcaagcttcctttcagttctctagcaatctgcagcaagcctcatttcagcatgacaaagagatgctgcaggctcagatagaggccacgaagaaactgcagcaagagatgatgaaagtgaagcaagcgatgctccttgaaggtggcttctttgagactgacgctgcgagaggcgccattaatgccccgatgacgaaagccctggactggtcagggaccaggtattgggctcctgatgcccgcaccactacatacaacgccggtcgcttttccactcctcaaccatcaggggcactcccaggacgggcgaatctgagggatgctgtgcccgcaagaggcagcagctccaagagcagtaacagctcaaccgctacaagcgtgtacagcaaccagacgacctcaacccggttgggctccacagccgtatctggtacctctgtgagctctttcccctctactgcccgtactcgaagttgggtggaagatcagtcccggaatctctctcccagtatgcgtggggctcacaacatcagcttcgttaccccaccctctagtcgctcaagtagccagggcacagtgtccacagtccctaaagaagtcctggactcctggacaggggcctttaacaccagaaggcagcctctcttcgcccacattaggaagagaggggagagtcgagcataatgagcgcgcagcgcttagacgtctcgcgatcgatactagtacaacctaaatccattataaaaaacttaggagcaaagtgattgcctcccaaggtccacaatgacagagacctacgacttcgacaagtcggcatgggacatcaaagggtcgatcgctccgatacaacccaccacctacagtgatggcaggctggtgccccaggtcagagtcatagatcctggtctaggcgacaggaaggatgaatgctttatgtacatgtttctgctgggggttgttgaggacagcgattccctagggcctccaatcgggcgagcatttgggttcctgcccttaggtgttggcagatccacagcaaagcccgaaaaactcctcaaagaggccactgagcttgacatagttgttagacgtacagcagggctcaatgaaaaactggtgttctacaacaacaccccactaactctcctcacaccttggagaaaggtcctaacaacagggagtgtcttcaacgcaaaccaagtgtgcaatgcggttaatctgataccgctcgataccccgcagaggttccgtgttgtttatatgagcatcacccgtctttcggataacgggtattacaccgttcctagaagaatgctggaattcagatcggtcaatgcagtggccttcaacctgctggtgacccttaggattgacaaggcgataggccctgggaagatcatcgacaatacagagcaacttcctgaggcaacatttatggtccacatcgggaacttcaggagaaagaagagtgaagtctactctgccgattattgcaaaatgaaaatcgaaaagatgggcctggtttttgcacttggtgggatagggggcaccagtcttcacattagaagcacaggcaaaatgagcaagactctccatgcacaactcgggttcaagaagaccttatgttacccgctgatggatatcaatgaagaccttaatcgattactctggaggagcagatgcaagatagtaagaatccaggcagttttgcagccatcagttcctcaagaattccgcatttacgacgacgtgatcataaatgatgaccaaggactattcaaagttctgtagaccgtagtgcccagcaatgcccgaaaacgacccccctcacaatgacagccagaaggcccggacaaaaaagccccctccgaaagactccacggaccaagcgagaggccagccagcagccgacggcaagcgcgaacaccaggcggccccagcacagaacagccctgacacaaggccaccaccagccaccccaatctgcatcctcctcgtgggacccccgaggaccaacccccaaggctgcccccgatccaaaccaccaaccgcatccccaccacccccgggaaagaaacccccagcaattggaaggcccctccccctcttcctcaacacaagaactccacaaccgaaccgcacaagcgaccgaggtgacccaaccgcaggcatccgactccctagacagatcctctctccccggcaaactaaacaaaacttagggccaaggaacatacacacccaacagaacccagaccccggcccacggcgccgcgcccccaacccccgacaaccagagggagcccccaaccaatcccgccggctcccccggtgcccacaggcagggacaccaacccccgaacagacccagcacccaaccatcgacaatccaagacgggggggcccccccaaaaaaaggcccccaggggccgacagccagcaccgcgaggaagcccacccaccccacacacgaccacggcaaccaaaccagaacccagaccaccctgggccaccagctcccagactcggccatcaccccgcagaaaggaaaggccacaacccgcgcaccccagccccgatccggcggggagccacccaacccgaaccagcacccaagagcgatccccgaaggacccccgaaccgcaaaggacatcagtatcccacagcctctccaagtcccccggtctcctcctcttctcgaagggaccaaaagatcaatccaccacacccgacgacactcaactccccacccctaaaggagacaccgggaatcccagaatcaagactcatccaatgtccatcatgggtctcaaggtgaacgtctctgccatattcatggcagtactgttaactctccaaacacccaccggtcaaatccattggggcaatctctctaagataggggtggtaggaataggaagtgcaagctacaaagttatgactcgttccagccatcaatcattagtcataaaattaatgcccaatataactctcctcaataactgcacgagggtagagattgcagaatacaggagactactgagaacagttttggaaccaattagagatgcacttaatgcaatgacccagaatataagaccggttcagagtgtagcttcaagtaggagacacaagagatttgcgggagtagtcctggcaggtgcggccctaggcgttgccacagctgctcagataacagccggcattgcacttcaccagtccatgctgaactctcaagccatcgacaatctgagagcgagcctggaaactactaatcaggcaattgagacaatcagacaagcagggcaggagatgatattggctgttcagggtgtccaagactacatcaataatgagctgataccgtctatgaaccaactatcttgtgatttaatcggccagaagctcgggctcaaattgctcagatactatacagaaatcctgtcattatttggccccagtttacgggaccccatatctgcggagatatctatccaggctttgagctatgcgcttggaggagacatcaataaggtgttagaaaagctcggatacagtggaggtgatttactgggcatcttagagagcggaggaataaaggcccggataactcacgtcgacacagagtcctacttcattgtcctcagtatagcctatccgacgctgtccgagattaagggggtgattgtccaccggctagagggggtctcgtacaacataggctctcaagagtggtataccactgtgcccaagtatgttgcaacccaagggtaccttatctcgaattttgatgagtcatcgtgtactttcatgccagaggggactgtgtgcagccaaaatgccttgtacccgatgagtcctctgctccaagaatgcctccgggggtacaccaagtcctgtgctcgtacactcgtatccgggtcttttgggaaccggttcattttatcacaagggaacctaatagccaattgtgcatcaatcctttgcaagtgttacacaacaggaacgatcattaatcaagaccctgacaagatcctaacatacattgctgccgatcactgcccggtagtcgaggtgaacggcgtgaccatccaagtcgggagcaggaggtatccagacgctgtgtacttgcacagaattgacctcggtcctcccatatcattggagaggttggacgtagggacaaatctggggaatgcaattgctaagttggaggatgccaaggaattgttggagtcatcggaccagatattgaggagtatgaaaggtttatcgagcactagcatagtctacatcctgattgcagtgtgtcttggagggttgatagggatccccgctttaatatgttgctgcagggggcgttgtaacaaaaagggagaacaagttggtatgtcaagaccaggcctaaagcctgatcttacgggaacatcaaaatcctatgtaaggtcgctctgatcctctacaactcttgaaacacaaatgtcccacaagtctcctcttcgtcatcaagcaaccaccgcacccagcatcaagcccacctgaaattatctccggcttccctctggccgaacaatatcggtagttaatcaaaacttagggtgcaagatcatccacaatgtcaccacaacgagaccggataaatgccttctacaaagataacccccatcccaagggaagtaggatagtcattaacagagaacatcttatgattgatagaccttatgttttgctggctgttctgtttgtcatgtttctgagcttgatcgggttgctagccattgcaggcattagacttcatcgggcagccatctacaccgcagagatccataaaagcctcagcaccaatctagatgtaactaactcaatcgagcatcaggtcaaggacgtgctgacaccactcttcaaaatcatcggtgatgaagtgggcctgaggacacctcagagattcactgacctagtgaaattaatctctgacaagattaaattccttaatccggatagggagtacgacttcagagatctcacttggtgtatcaacccgccagagagaatcaaattggattatgatcaatactgtgcagatgtggctgctgaagagctcatgaatgcattggtgaactcaactctactggagaccagaacaaccaatcagttcctagctgtctcaaagggaaactgctcagggcccactacaatcagaggtcaattctcaaacatgtcgctgtccctgttagacttgtatttaggtcgaggttacaatgtgtcatctatagtcactatgacatcccagggaatgtatgggggaacttacctagtggaaaagcctaatctgagcagcaaaaggtcagagttgtcacaactgagcatgtaccgagtgtttgaagtaggtgttatcagaaatccgggtttgggggctccggtgttccatatgacaaactatcttgagcaaccagtcagtaatgatctcagcaactgtatggtggctttgggggagctcaaactcgcagccctttgtcacggggaagattctatcacaattccctatcagggatcagggaaaggtgtcagcttccagctcgtcaagctaggtgtctggaaatccccaaccgacatgcaatcctgggtccccttatcaacggatgatccagtgatagacaggctttacctctcatctcacagaggtgttatcgctgacaatcaagcaaaatgggctgtcccgacaacacgaacagatgacaagttgcgaatggagacatgcttccaacaggcgtgtaagggtaaaatccaagcactctgcgagaatcccgagtgggcaccattgaaggataacaggattccttcatacggggtcttgtctgttgatctgagtctgacagttgagcttaaaatcaaaattgcttcgggattcgggccattgatcacacacggttcagggatggacctatacaaatccaaccacaacaatgtgtattggctgactatcccgccaatgaagaacctagccttaggtgtaatcaacacattggagtggataccgagattcaaggttagtccctacctcttcactgtcccaattaaggaagcaggcgaagactgccatgccccaacatacctacctgcggaggtggatggtgatgtcaaactcagttccaatctggtgattctacctggtcaagatctccaatatgttttggcaacctacgatacttccagggttgaacatgctgtggtttattacgtttacagcccaagccgctcattttcttacttttatccttttaggttgcctataaagggggtccccatcgaattacaagtggaatgcttcacatgggaccaaaaactctggtgccgtcacttctgtgtgcttgcggactcagaatctggtggacatatcactcactctgggatggtgggcatgggagtcagctgcacagtcacccgggaagatggaaccaatcgcagatagggctgctagtgaaccaatcacatgatgtcacccagacatcaggcatacccactagtgtgaaatagacatcagaattaagaaaaacgtagggtccaagtggttccccgttatggactcgctatctgtcaaccagatcttataccctgaagttcacctagatagcccgatagttaccaataagatagtagccatcctggagtatgctcgagtccctcacgcttacagcctggaggaccctacactgtgtcagaacatcaagcaccgcctaaaaaacggattttccaaccaaatgattataaacaatgtggaagttgggaatgtcatcaagtccaagcttaggagttatccggcccactctcatattccatatccaaattgtaatcaggatttatttaacatagaagacaaagagtcaacgaggaagatccgtgaactcctcaaaaaggggaattcgctgtactccaaagtcagtgataaggttttccaatgcttaagggacactaactcacggcttggcctaggctccgaattgagggaggacatcaaggagaaagttattaacttgggagtttacatgcacagctcccagtggtttgagccctttctgttttggtttacagtcaagactgagatgaggtcagtgattaaatcacaaacccatacttgccataggaggagacacacacctgtattcttcactggtagttcagttgagttgctaatctctcgtgaccttgttgctataatcagtaaagagtctcaacatgtatattacctgacatttgaactggttttgatgtattgtgatgtcatagaggggaggttaatgacagagaccgctatgactattgatgctaggtatacagagcttctaggaagagtcagatacatgtggaaactgatagatggtttcttccctgcactcgggaatccaacttatcaaattgtagccatgctggagcctctttcacttgcttacctgcagctgagggatataacagtagaactcagaggtgctttccttaaccactgctttactgaaatacatgatgttcttgaccaaaacgggttttctgatgaaggtacttatcatgagttaactgaagctctagattacattttcataactgatgacatacatctgacaggggagattttctcatttttcagaagtttcggccaccccagacttgaagcagtaacggctgctgaaaatgttaggaaatacatgaatcagcctaaagtcattgtgtatgagactctgatgaaaggtcatgccatattttgtggaatcataatcaacggctatcgtgacaggcacggaggcagttggccaccgctgaccctccccctgcatgctgcagacacaatccggaatgctcaagcttcaggtgaagggttaacacatgagcagtgcgttgataactggaaatcttttgctggagtgaaatttggctgctttatgcctcttagcctggatagtgatctgacaatgtacctaaaggacaaggcacttgctgctctccaaagggaatgggattcagtttacccgaaagagttcctgcgttacgaccctcccaagggaaccgggtcacggaggcttgtagatgttttccttaatgattcgagctttgacccatatgatgtgataatgtatgttgtaagtggagcttacctccatgaccctgagttcaacctgtcttacagcctgaaagaaaaggagatcaaggaaacaggtagactttttgctaaaatgacttacaaaatgagggcatgccaagtgattgctgaaaatctaatctcaaacgggattggcaaatattttaaggacaatgggatggccaaggatgagcacgatttgactaaggcactccacactctagctgtctcaggagtccccaaagatctcaaagaaagtcacaggggggggccagtcttaaaaacctactcccgaagcccagtccacacaagtaccaggaacgtgagagcagcaaaagggtttatagggttccctcaagtaattcggcaggaccaagacactgatcatccggagaatatggaagcttacgagacagtcagtgcatttatcacgactgatctcaagaagtactgccttaattggagatatgagaccatcagcttgtttgcacagaggctaaatgagatttacggattgccctcatttttccagtggctgcataagaggcttgagacctctgtcctgtatgtaagtgaccctcattgcccccccgaccttgacgcccatatcccgttatataaagtccccaatgatcaaatcttcattaagtaccctatgggaggtatagaagggtattgtcagaagctgtggaccatcagcaccattccctatctatacctggctgcttatgagagcggagtaaggattgcttcgttagtgcaaggggacaatcagaccatagccgtaacaaaaagggtacccagcacatggccctacaaccttaagaaacgggaagctgctagagtaactagagattactttgtaattcttaggcaaaggctacatgatattggccatcacctcaaggcaaatgagacaattgtttcatcacatttttttgtctattcaaaaggaatatattatgatgggctacttgtgtcccaatcactcaagagcatcgcaagatgtgtattctggtcagagactatagttgatgaaacaagggcagcatgcagtaatattgctacaacaatggctaaaagcatcgagagaggttatgaccgttaccttgcatattccctgaacgtcctaaaagtgatacagcaaattctgatctctcttggcttcacaatcaattcaaccatgacccgggatgtagtcatacccctcctcacaaacaacgacctcttaataaggatggcactgttgcccgctcctattggggggatgaattatctgaatatgagcaggctgtttgtcagaaacatcggtgatccagtaacatcatcaattgctgatctcaagagaatgattctcgcctcactaatgcctgaagagaccctccatcaagtaatgacacaacaaccgggggactcttcattcctagactgggctagcgacccttactcagcaaatcttgtatgtgtccagagcatcactagactcctcaagaacataactgcaaggtttgtcctgatccatagtccaaacccaatgttaaaaggattattccatgatgacagtaaagaagaggacgagggactggcggcattcctcatggacaggcatattatagtacctagggcagctcatgaaatcctggatcatagtgtcacaggggcaagagagtctattgcaggcatgctggataccacaaaaggcttgattcgagccagcatgaggaagggggggttaacctctcgagtgataaccagattgtccaattatgactatgaacaattcagagcagggatggtgctattgacaggaagaaagagaaatgtcctcattgacaaagagtcatgttcagtgcagctggcgagagctctaagaagccatatgtgggcgaggctagctcgaggacggcctatttacggccttgaggtccctgatgtactagaatctatgcgaggccaccttattcggcgtcatgagacatgtgtcatctgcgagtgtggatcagtcaactacggatggttttttgtcccctcgggttgccaactggatgatattgacaaggaaacatcatccttgagagtcccatatattggttctaccactgatgagagaacagacatgaagcttgccttcgtaagagccccaagtcgatccttgcgatctgctgttagaatagcaacagtgtactcatgggcttacggtgatgatgatagctcttggaacgaagcctggttgttggctaggcaaagggccaatgtgagcctggaggagctaagggtgatcactcccatctcaacttcgactaatttagcgcataggttgagggatcgtagcactcaagtgaaatactcaggtacatcccttgtccgagtggcgaggtataccacaatctccaacgacaatctctcatttgtcatatcagataagaaggttgatactaactttatataccaacaaggaatgcttctagggttgggtgttttagaaacattgtttcgactcgagaaagataccggatcatctaacacggtattacatcttcacgtcgaaacagattgttgcgtgatcccgatgatagatcatcccaggatacccagctcccgcaagctagagctgagggcagagctatgtaccaacccattgatatatgataatgcacctttaattgacagagatgcaacaaggctatacacccagagccataggaggcaccttgtggaatttgttacatggtccacaccccaactatatcacattttagctaagtccacagcactatctatgattgacctggtaacaaaatttgagaaggaccatatgaatgaaatttcagctctcataggggatgacgatatcaatagtttcataactgagtttctgctcatagagccaagattattcactatctacttgggccagtgtgcggccatcaattgggcatttgatgtacattatcatagaccatcagggaaatatcagatgggtgagctgttgtcatcgttcctttctagaatgagcaaaggagtgtttaaggtgcttgtcaatgctctaagccacccaaagatctacaagaaattctggcattgtggtattatagagcctatccatggtccttcacttgatgctcaaaacttgcacacaactgtgtgcaacatggtttacacatgctatatgacctacctcgacctgttgttgaatgaagagttagaagagttcacatttctcttgtgtgaaagcgacgaggatgtagtaccggacagattcgacaacatccaggcaaaacacttatgtgttctggcagatttgtactgtcaaccagggacctgcccaccaattcgaggtctaagaccggtagagaaatgtgcagttctaaccgaccatatcaaggcagaggctatgttatctccagcaggatcttcgtggaacataaatccaattattgtagaccattactcatgctctctgacttatctccggcgaggatcgatcaaacagataagattgagagttgatccaggattcattttcgacgccctcgctgaggtaaatgtcagtcagccaaagatcggcagcaacaacatctcaaatatgagcatcaaggctttcagacccccacacgatgatgttgcaaaattgctcaaagatatcaacacaagcaagcacaatcttcccatttcagggggcaatctcgccaattatgaaatccatgctttccgcagaatcgggttgaactcatctgcttgctacaaagctgttgagatatcaacattaattaggagatgccttgagccaggggaggacggcttgttcttgggtgagggatcgggttctatgttgatcacttataaagagatacttaaactaaacaagtgcttctataatagtggggtttccgccaattctagatctggtcaaagggaattagcaccctatccctccgaagttggccttgtcgaacacagaatgggagtaggtaatattgtcaaagtgctctttaacgggaggcccgaagtcacgtgggtaggcagtgtagattgcttcaatttcatagttagtaatatccctacctctagtgtggggtttatccattcagatatagagaccttgcctgacaaagatactatagagaagctagaggaattggcagccatcttatcgatggctctgctcctgggcaaaataggatcaatactggtgattaagcttatgcctttcagcggggattttgttcagggatttataagttatgtagggtctcattatagagaagtgaaccttgtataccctagatacagcaacttcatctctactgaatcttatttggttatgacagatctcaaggctaaccggctaatgaatcctgaaaagattaagcagcagataattgaatcatctgtgaggacttcacctggacttataggtcacatcctatccattaagcaactaagctgcatacaagcaattgtgggagacgcagttagtagaggtgatatcaatcctactctgaaaaaacttacacctatagagcaggtgctgatcaattgcgggttggcaattaacggacctaagctgtgcaaagaattgatccaccatgatgttgcctcagggcaagatggattgcttaattctatactcatcctctacagggagttggcaagattcaaagacaaccaaagaagtcaacaagggatgttccacgcttaccccgtattggtaagtagcaggcaacgagaacttatatctaggatcacccgcaaattctgggggcacattcttctttactccgggaacaaaaagttgataaataagtttatccagaatctcaagtccggctatctgatactagacttacaccagaatatcttcgttaagaatctatccaagtcagagaaacagattattatgacggggggtttgaaacgtgagtgggtttttaaggtaacagtcaaggagaccaaagaatggtataagttagtcggatacagtgccctgattaaggactaattggttgaactccggaaccctaatcctgccctaggtggttaggcattatttgcaatatattaaagaaaactttgaaaatacgaagtttctattcccagctttgtctggt

SEQ ID NO: 11 - protein, 540 aa, Norovirus
MKMASNDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPILTVEEMTNSRFPIPLEKLFTGPSSTFVVQPQNGRCTTDGVLLGTTQLSPVNICTFRGDVTHITGSRNYTMNLASQNWNSYDPTEEIPAPLGTPDFVGKIQGVLTQTTRTDGSTRGHKATVYTGSADFSPKLGRVQFATDTDNDFDTNQNTKFTPVGVIQDGGTTHRNEPQQWVLPSYSGRNTHNVHLAPAVAPTFPGEQLLFFRSTMPGSSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAV

SEQ ID NO: 12 - protein, 268 aa, Norovirus
MAGAFFAGLASDVLSSGLGSLINAGAGAINQTVEFENNRKLQQASFQFSSNLQQASFQHDKEMLQSQIDATKKLQQEMMRVKQAMLLEGGFSETDAARGAINAPMTKVLDWSGTRYWAPDARTTTYNAGRFSTPQSSGAPPGRANLRAAVPAQGSSSTSSNSSIATSMYSNQTTSTRLGSTAGSGTSVSSLPSSARTRSWVEDQNRNLSPFMRGAHNISFVTPPSSRSSSQGTVSTVPKEVLDSWTGAFNTRRQPLFAHIRRRGESRV

SEQ ID NO: 13 - protein, 540 aa, Norovirus
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWSNYDPTEETPAPLGTPDFVGKIQGVLTQTTRTDGSTRGHKATVYTGSADFAPKLGRVQFETDTDRDFEANQNTKFTPVGVIQDGSTTHRNEPQQWVLPSYSGRNTPNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAL

SEQ ID NO: 14 - protein, 268 aa, Norovirus
MAGAFFAGLASDVLGSGLGSLINAGAGAINQKVEFENNRKLQQASFQFSSNLQQASFQHDKEMLQAQIEATKRLQQEMMKVKQAMLLEGGFSETDAARGAINAPMTKALDWSGTRYWAPDARTTTYNAGRFSTPQPSGALPGRANLRDAVPARGSSSKSSNSSTATSVYSNQTTSTRLGSTAGSGTSVSSFPSTARTRSWVEDQSRNLSPFMRGAHNISFVTPPSSRSSSQGTVSTVPKEILDSWTGAFNTRRQPLFAHIRKRGESRA

SEQ ID NO: 15 - protein, 540 aa, Norovirus
MKMASSDANPSDGSAANLVPEVNNEVMALEPVVGAAIAAPVAGQQNVIDPWIRSNFVQAPGGEFTVSPRNAPGEILWSAPLGPDLNPYLSHLARMYNGYAGGFEVQVILAGNAFTAGKVIFAAVPPNFPTEGLSPSQVTMFPHIVVDVRQLEPVLIPLPDVRNNFYHYNQSNDPTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWSNYDPTEEIPAPLGTPDFVGKIQGVLTQTTRTDGSTRGHKATVYTGSANFAPKLGRVQFETDTDHDFEANQNTKFTPVGVIQDGSTTHRNEPQQWVLPSYSGRNTPNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRAL

SEQ ID NO: 16 - protein, 268 aa, Norovirus
MAGAFFAGLASDVLGSGLGSLINAGAGAINQKVEFENNRKLQQASFQFSSNLQQASFQHDKEMLQAQIEATKRLQQEMMKVKQAMLLEGGFSETDAARGAINAPMTKALDWSGTRYWAPDARTTTYNAGRFSTPQPSGALLGRANLRDAVPARGSSSKSTNSSTATSVYSNQTTSTRLGSTAGSGTSVSSFPSTARTRSWVEDQSRNLSPFMRGAHNISFVTPPSSRSSSQGTVSTVPKEILDSWTGAFNTRRQPLFAHIRKRGESRA

SEQ ID NO: 17 - DNA, 68 bp, Artificial Sequence
  ATU
tagcctaccctccatcattgttataaaaaacttaggaaccaggtccacacagccgccagcccatcaac

SEQ ID NO: 18 - DNA, 25 bp, Artificial Sequence
  ATU
agcgcttagacgtctcgcgatcgat

(19) United States
(12) Patent Application Publication
(10) Pub. No.: US 2022/0143175 A1
(43) Pub. Date: May 12, 2022

(54) NOROVIRUS VACCINES

(71) Applicant: Themis Bioscience GmbH, Vienna (AT)

(72) Inventors: Erich Tauber, Muckendorf (AT); Matthias Müllner, Pixendorf (AT); Katrin Ramsauer, Vienna (AT); Sabrina Schrauf, Mörbisch Am See (AT); Angelika Irmler, Vienna (AT); Patrick Csar, Königstetten (AT); David A. Maier, Vienna (AT); Jochen Stritzker, Kierling (AT)

(21) Appl. No.: 17/418,197

(22) PCT Filed: Dec. 30, 2019
(86) PCT No.: PCT/EP2019/087160

§ 371 Date: Jun. 24, 2021

(30) Foreign Application Priority Data
Dec. 28, 2018 (EP) 18248201.8

(51) Int. Cl. A61K 39/295; A61P 31/14; C12N 7/00

(52) U.S. Cl. CPC A61K 39/295; C12N 7/00; A61P 31/14

(57) ABSTRACT

The present invention provides immunogenic compositions, nucleic acid molecules and VLPs suitable as Norovirus vaccine candidates. Further provided are host cells for producing the biological material as well as methods for producing and/or purifying the immunogenic compositions and VLPs. Further provided is an immunogenic composition for use in methods of preventing/treating a Norovirus infection in a subject.

### TECHNICAL FIELD

The present invention provides immunogenic compositions, nucleic acid molecules and VLPs suitable as Norovirus vaccine candidates. In particular, suitable antigens originating from relevant Norovirus genotypes, suitable vaccine architectures and methods of efficiently producing said antigens in a suitable vector backbone are provided. Further provided are host cells for producing the biological material as well as methods for producing and/or purifying the immunogenic compositions and VLPs. Further provided is an immunogenic composition for use in a method of preventing/treating a Norovirus infection in a subject.

### BACKGROUND OF INVENTION

Human Noroviruses are the leading cause of acute gastroenteritis worldwide and are replacing rotavirus as predominant gastrointestinal pathogen in the pediatric population (Ramani et al., Curr. Opin. Gastroenterol., 2014). The virus causes acute debilitating illness characterized by vomiting and diarrhea as typical symptoms of a Norovirus infection. The infections can occur in all age groups and result in significant morbidity and mortality, particularly in the very old and the very young. Approximately 200,000 children per year die from complications of Norovirus infections worldwide according to an estimation. The transmission rate is very high, requiring a very low infectious dose of only less than 10-100 virions. The disease is mostly self-limiting within a few days. Nevertheless, the virus can cause also severe outcomes mainly in elderly, young children and immunosuppressed groups. Beyond supportive care no treatment is currently available.

Norovirus is divided into five genogroups (I-V), with GI, GII and GIV causing human infections. Each genogroup is further subdivided into genotypes based on analysis of the amino acid sequence of its major viral capsid protein VP1. The Norwalk virus, the prototype human Norovirus is classified as a GI virus.

The epidemiology of the virus is very complex with over 30 genotypes in three genogroups (GI, GII and GIV) causing infections in humans. The division into genotypes is based on the analysis of the amino acid sequences of its major viral capsid protein VP1. Most confirmed infections are associated with genotype GII.4, i.e. over 80% (WHO, Status of Vaccine Research and Development for Norovirus, prepared for WHO PD-VAC, 2014). A subdivision into serotypes as commonly done for virus is impossible due to the fact that the virus cannot be cultured in vitro. Therefore, it remains to be elucidated if this classification is of biological relevance. Further, the inability to culture Norovirus hampers research on pathogenesis, vaccine development, and diagnostics. Finally, epidemiology of Norovirus on a global level is highly dynamic and seasonality and predominance of certain Norovirus strains varies, also depending on the prevailing climate conditions.

Structurally, noroviruses are ˜38 nm icosahedral viruses with an ˜7.5-kb single-stranded, positive-sense RNA genome that encodes three large open reading frames (ORFs). ORF1 encodes the replicase polyprotein, while ORFs 2 and 3 encode the major and minor capsid proteins VP1 and VP2, respectively. Expression of the major capsid protein VP1 (ORF2) in baculovirus and Venezuelan equine encephalitis (VEE) virus was shown to result in formation of virus-like particles (VLPs) composed of 180 copies of the monomeric protein. The monomer is structurally divided into the shell domain (S) that forms the core of the particle and the protruding domain (P) that extends away from the core. The P domain is further subdivided into the P1 subdomain (residues 226 to 278 and 406 to 520) and the P2 subdomain (residues 279 to 405) (53). P2 represents the most exposed surface of the viral particle and determines interaction with both potential neutralizing antibody and histo-blood group antigens (HBGAs) (Lindesmith et al., Norovirus GII.4 Strain Antigenic Variation, Journal of Virology, vol. 85, no. 1, 2011).

Another fact complicating vaccine development is that the virus rapidly undergoes mutations, antigenic drift and recombination events. Antigenic drift is a typical feature of noroviruses belonging to the GII.4 genotype. In the early 2000s, the association of host genetics and the risk of Norovirus infection was identified. Pathogenesis of this virus is mainly driven by its binding capacity to human histo-blood group antigens (HBGA). These glycans are cell attachment factors for Noroviruses and are present on epithelial cell surfaces (mainly the gut and respiratory tract). Different variants of HBGAs exist. Human challenge studies have demonstrated that only individuals expressing certain HBGAs are susceptible to infection with the prototype virus Norwalk virus belonging to GI. Interestingly, Norovirus GII.4 binds a variety of HBGAs, possibly explaining its predominance in causing Norovirus infections and outbreaks.

Noroviruses are non-enveloped single-stranded, positive-sense RNA viruses and belong to the Family of Caliciviridae. The capsid is formed by two proteins, the major capsid protein VP1 and the minor capsid protein VP2. The outer capsid shell consists of the major capsid protein VP1. This protein has the ability to self-assemble into virus-like particles (VLPs), which morphologically and antigenically resemble the native virus. Therefore, for the specific case of Norovirus, VLPs can be highly immunogenic and seem to represent a promising candidate for vaccine development provided that a technique can be established allowing that functional recombinant VLPs can be produced and further provided that said VLPs will present the correct antigens able to elicit a desired immune response. No additional protein is needed. Nevertheless, the minor capsid protein if expressed together with VP1 can also be incorporated into the virus-like particles. It stays within the capsid and is thus not target of the immune response. Despite evidence that at least GI Norwalk virus minor structural protein VP2 is present in infectious virions, the available crystallographic and electron cryomicroscopy structures of NV have not revealed the location of VP2. Later, it was determined that VP1 associates with VP2 at the interior surface of the capsid, specifically with the shell (S) domain of VP1. Studies have shown that VP2 may increase the stability of the VLPs, doubling the half-life, at least in vitro, which was demonstrated for VP2 of the GI Norwalk Norovirus in an analytical setting (Vongpunsawad et al., Norwalk Virus Minor Capsid Protein VP2 Associates within the VP1 Shell Domain, J. Virol., 2013, 87:94818-4825). Notably, VP2 is not essential for Norovirus VLP formation, which may be a reason for the fact that so far no efforts have been made to design a vaccine based on the additional protein VP2.

Currently, there are no licensed vaccines available. Given the high mortality and morbidity of the virus, the need for a prophylactic Norovirus vaccine is very high. The facts that the virus cannot be grown in cell culture as well as that no appropriate preclinical model system exist to explore pathogenesis and vaccine efficacy hamper vaccine development. A few vaccine candidates are currently in development, mainly based on virus-like particles. Virus-like particles represent the best way to move forward in the vaccine development as they are highly immunogenic and easy to produce due to the self-assemble function of the VP1 protein. Most of these vaccine candidates are in pre-clinical development, except one.

Vaccine feasibility was recently demonstrated with this candidate by showing protection particularly against severe disease in two human challenge studies. This most advanced vaccine candidate is a bivalent virus-like particle vaccine against genotypes G1.1/GII.4 based on a consensus sequence for GII.4. These particles are formed solely by the expression of the major capsid protein VP1. In all reported activities in Norovirus vaccine development, however, no VP2 was expressed.

The most advanced vaccine candidate for Norovirus currently in development was developed by Takeda Vaccines and is a recombinant VLP vaccine achieved by the expression of the major capsid protein VP1 solely. The vaccine is based on the VP1 sequence of GI.1 Norwalk virus and a VP1 consensus sequence of three GII.4 viruses. A bivalent vaccine candidate. The VLPs are expressed in the baculovirus system. Purified VLPs resemble the vaccine candidate. Thus, the vaccine can be classified as a recombinant protein vaccine. In order to make such protein vaccines potent in human beings, the protein has to be formulated with additional adjuvants like Alum and MPL, increasing also the reactogenicity of the vaccine candidate. An alternative VLP candidate is being developed by Arizona State University using the same construct as the Takeda VLP, but it is produced in a plant-based vector. A Norovirus VLP-rotavirus protein combination vaccine candidate is currently in preclinical development, and clinical trials may begin in the near term (see WHO, 2014, supra). The vaccine is being co-developed by the University of Tampere (Finland) and UMN Pharma (Japan). Finally, the University of Cincinnati has conducted preclinical immunological studies on a Norovirus P particle construct. This vaccine candidate is derived from the protruding (P) domain of the Norovirus VP1 capsid protein (see WHO, 2014, supra). Kim et al. (Virology, 484 (2015) 163-169) discloses the generation of recombinant Newcastle disease virus vectors expressing a naturally occurring open reading frame (ORF) 2 and 3 including the 3″-untranslated region (UTR) of one specific isolate of Norovirus, namely Norwalk virus Hu/NV/Norwalk virus/1968/US). Kim et al. is silent as to an artificial vaccine insert construct for artificially connecting, expressing, and combining Norovirus sequences suitable as antigens.

Regarding Norovirus structure, most of the work done has been established in Norwalk virus, in particular using VP1. Depending on whether full-length or successively truncated recombinant forms of VP1 were used, structurally different Norovirus complexes were observed, i.e. (A) Virus-like particles (180-mer: ˜37 nm); (B) S particles (180-mer: ˜27 nm); (C) P particles (24-mer: ˜20 nm), i.e. those particles only containing the protruding domains of the capsid; (D) small P particles (12-mer: ˜14 nm); (E) P dimers (˜6 nm), or (F) linear structures of norovirus VP1 comprising truncated S (1-217) and P (226-530) domains that are linked by a short hinge. Numbers are based on Norwalk virus VP1 (Tan et al., Terminal modifications of norovirus P domain resulted in a new type of subviral particles, the small P particles, Virology. 2011 Feb. 20; 410(2):345-52).

From a lesser-developed country perspective, there are unique issues to consider for vaccine development and feasibility. Dose number and schedule are important, as well as possibly different circulating strains compared to those found in developed countries. Furthermore, the current vaccine approach for an indication in adults is based on an intramuscular injection of the vaccine with an effective immune response that is thought in part to rely on the boosting of memory from previous natural infection. As a Norovirus vaccine would likely be targeted to younger children, the effectiveness of the vaccine in a naïve infant may be less, and an alternative mucosal priming-parenteral boost hypothetically may be necessary, but also complicates the development of a vaccine for use in a resource-limited setting (see WHO, 2014, supra).

Taking the present status quo of Norovirus vaccine development into account, it was thus an object of the present invention to establish a Norovirus vaccine candidate based on the expression of two proteins, VP1 and VP2, to confer broad protection against the most common and severe Norovirus infection genotypes. It should be figured out whether co-expression of both proteins in a suitable vector system could induce a better immunogenicity in a target population.

In particular, it was an object to establish the potential of VP2-based vaccines so far not being studied in detail as protective Norovirus antigenic sequences. Based on this co-expression strategy, a platform to rapidly develop vaccine candidates should be established regarding the implications of antigenic diversity and drift in the context of Norovirus to be able to provide efficacious vaccine compositions readily adaptable to the norovirus antigenic variation. Furthermore, stable and immunogenic VP1-based VLP vaccines should be created. This strategy should also address the high alternating predominance of different Norovirus GII.4 and also non-GII.4 genotypes in view of the fact that Norovirus variations on a global level demands the design of different vaccines following the same basic construction principle to obtain a preventive measure at short notice. To this end, the inventors also aimed and identifying consensus sequences of VP1 and VP2 Norovirus sequences to establish sequences with a high or low degree of variation over several Norovirus genogroups and genotypes to establish a vaccine with broad coverage against different Noroviruses. Thus, a vaccine candidate based on the co-expression of VP1 and VP2 should be established to provide more stable VLPs, in turn increasing the immunogenicity of the vaccine candidate. The strategy of co-expression was also designed to be used for the rapid development of multivalent and seasonal Norovirus vaccine candidates.

Furthermore, it was an object to provide a safe and efficient manufacturing technique avoiding the need of adjuvants and further allowing the provision of a vaccine which can be administered to children. Finally, a reliable expression vector system should be provided that allows high yield vaccine production, and thus could represent a manufacturing advantage through relatively low cost of goods whilst simultaneously being safe and efficient in antigen presentation. In addition, the proteins expressed by a self-replicating and therefore self-adjuvanting vector system itself should be used to increase the potency of the vaccine candidate.

### SUMMARY OF THE INVENTION

The above objects have been achieved by providing, in a first aspect, an immunogenic composition comprising at least one recombinant Norovirus antigen, and/or a sequence encoding the same, wherein the at least one recombinant Norovirus antigen is encoded by at least one nucleic acid sequence encoding at least one Norovirus VP1 sequence and at least one Norovirus VP2 sequence, wherein the at least one nucleic acid sequence is operably linked to a vector backbone, wherein the immunogenic composition optionally comprises at least one pharmaceutically acceptable carrier and/or excipient, optionally wherein the Norovirus VP1 sequence and/or the Norovirus VP2 sequence is independently derived from a Norovirus of the GII genogroup, preferably from a Norovirus having a GII.4 genotype, preferably wherein the Norovirus VP1 sequence and the Norovirus VP2 sequence are encoded by the same nucleic acid sequence and are optionally separated by at least one linker sequence.

In one embodiment, the vector backbone is selected from the group consisting of a measles virus backbone, a lentivirus backbone, a Vesicular stomatitis virus (VSV) backbone, or a vaccinia Ankara backbone, preferably wherein the vector backbone is from a recombinant live-attenuated measles virus strain, the measles virus strain preferably being selected from the group consisting of a Schwarz strain, a Zagreb strain, an AIK-C strain and a Moraten strain, and/or the VP1 and/or the VP2 sequence are independently selected from a Norovirus having a GII.4 genotype, preferably from a sequence from a Norovirus Hu/GII.4/Sydney/NSW0514/2012/AU, a Norovirus Hu/GII.4/Orange/NSW001P/2008/AU, a Norovirus Hu/GII.4/NIHIC17.5/2012/USA, a Norovirus Hu/GII.4/G114-NG1242/2011/JP, or a Norovirus Hu/GII.4/Hiroshima4/2012/JP, wherein the VP1 and the VP2 sequence are derived from the same Norovirus, or wherein the VP1 and the VP2 sequence are derived from a different Norovirus, optionally wherein the VP1 and the VP2 sequence are modified in comparison to the reference sequence.

In another embodiment, the at least one VP1 and/or the at least one VP2 sequence represent(s) a conserved consensus sequence, and/or the immunogenic composition is a bivalent or a multivalent immunogenic composition, wherein the at least one nucleic acid sequence encoding the at least one Norovirus VP1 sequence and/or the at least one nucleic acid sequence encoding at least one Norovirus VP2 sequence are selected from at least two different Norovirus genogroups, preferably a GII and a GI genogroups, and/or wherein the at least one nucleic acid sequence encoding the at least one Norovirus VP1 sequence and/or the at least one nucleic acid sequence encoding at least one Norovirus VP2 sequence are selected from different Norovirus GII genotypes, preferably a GII.4 and a GII.17 or GII.2 genotype.

In a second aspect, there is provided a nucleic acid molecule comprising at least a first and at least a second nucleic acid sequence, wherein the first nucleic acid sequence encodes at least one VP1 protein of a Norovirus or a functional fragment thereof, and wherein the second nucleic acid sequence encodes at least one VP2 protein of a Norovirus or a functional fragment thereof, optionally wherein the at least one first and the at least one second nucleic acid sequence is/are separated by at least one linker, optionally wherein the nucleic acid molecule comprises a sequence selected from any one of SEQ ID NOs:3 to 8, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or wherein the first nucleic acid sequence and/or the second nucleic acid sequence represent(s) a conserved consensus sequence.

In a third aspect, there is provided a recombinant virus-like particle (VLP) comprising at least one recombinant Norovirus VP1 antigen and at least one recombinant Norovirus VP2 antigen.

In yet another aspect, there is provided a host cell comprising at least one nucleic acid sequence encoding at least one Norovirus VP1 sequence and at least one Norovirus VP2 sequence, or comprising at least one nucleic acid molecule according to the second aspect, or comprising at least one nucleic acid sequence encoding a recombinant virus-like particle (VLP) according the above third aspect, preferably wherein the at least one host cell is selected from the group consisting of Vero cells, chicken embryo fibroblast cells, HEK293 cells, HeLa cells, fetal rhesus lung cells or MRC5 cells.

In a further aspect, there is provided a method for producing an immunogenic composition according to the first aspect, or for producing a recombinant virus-like particle (VLP) according to the third aspect, the method comprising the following steps: (i) inserting at least one nucleic acid sequence as defined according to the first aspect, or inserting at least one nucleic acid molecule as defined according to the second aspect above into a vector backbone as defined in the turn of the first aspect to operably link the nucleic acid sequence or molecule and the vector backbone to obtain a recombinant chimeric virus sequence; (ii) infecting at least one host cell with at least one recombinant chimeric virus sequence obtained in step (i) to obtain a virus sample; (iii) optionally: clarifying the virus sample of step (ii); (iv) optionally: purifying the clarified virus sample of step (iii); (v) optionally: formulating the at least one recombinant chimeric virus with at least one pharmaceutically and/or veterinary acceptable carrier and/or excipient; (vi) obtaining an immunogenic composition comprising infectious virus particles and virus-like particles (VLPs); (vii) optionally: further purifying the immunogenic composition and thus obtaining, separated from each other, recombinant virus-like particles (VLPs) and/or infectious virus particles.

In a further aspect, there is provided an immunogenic composition according to the first aspect, or a recombinant virus-like particle (VLP) according to the third aspect for use in a method of preventing or treating a Norovirus virus disease in a subject.

The present invention will now be described in detail based on the detailed description, including non-limiting Examples, the attached drawings and the sequences as provided with the sequence listing.

### BRIEF DESCRIPTION OF DRAWINGS

FIG. 1 (FIGS. 1A to B) shows in FIG. 1A an alignment of VP1 amino acid sequences of SEQ ID Nos:1, 11, 13 and 15 of their entire length. The alignment was created using the publicly available tool MUSCLE (MUltiple Sequence Comparison by Log-Expectation; https://www.ebi.ac.uk/Tools/msa/muscle/) using default parameters. Asterisks (*) indicate positions which have a single, fully conserved residue. Colons (:) indicate conservation between groups of strongly similar properties and thus highly conserved residues. Periods (.) indicate conservation between groups of weakly similar properties. The raw data were then immediately submitted to the Tool MView (version 1.63), which was also used according to default parameters (https://www.ebi.ac.uk/Tools/msa/mview/; Brown, N. P., Leroy C., Sander C. (1998). MView: A Web compatible database search or multiple alignment viewer. Bioinformatics. 14 (4):380-381). The result is shown in FIG. 1B. The upper four rows represent the sequence of the corresponding SEQ ID NOs. consensus 100%, 90%, 80% and 70% then show the default calculated consensus mechanism displaying consensus lines calculated at four levels of identity (100%, 90%, 80%, 70%). The default protein alignment is defined by physicochemical (positive, aromatic etc.) and sterical (small, tiny etc.) properties of the respective amino acid sequences (symbols: classical amino acid one-letter codes, further symbols are: alcohol=>o {S, T}; aliphatic=>I {I, L, V}; aromatic=>a {F, H, W, Y}; charged=>c {D, E, H, K, R}; hydrophobic=>h {A, C, F, G, H, I, K, L, M, R, T, V, W, Y}; negative=>−{D, E}; polar=>p {C, D, E, H, K, N, Q, R, S, T}; positive=>+{H, K, R}; small=>s {A, C, D, G, N, P, S, T, V}; tiny=>u {A, G, S}; turnlike=>t {A, C, D, E, G, H, K, N, Q, R, S, T}; stop=>* {*}.

FIG. 2 (FIGS. 2A and B) shows in FIG. 2A an alignment of VP2 amino acid sequences of SEQ ID Nos:2, 12, 14 and 16 of their entire length. The alignment was created using the publicly available tool MUSCLE using default parameters. FIG. 2B shows a MView 1.63 consensus pattern definition (default parameters used) for the given VP2 sequences as detailed for FIG. 1 above. Based on at least two, preferably at least three given input sequences (DNA or protein) the skilled person can thus easily identify a consensus pattern as described in and disclosed with FIGS. 1 and 2.

FIG. 3 shows Norovirus VLP expression in MV-Noro infected cells. Cells were infected with a MOI of 0.01 and three days post infection, the supernatant was removed, the cells were washed and fixed. Subsequently, the fixed cells were incubated with a Norovirus GII.4 antibody recognizing Norovirus virus-like particle (VLP) from GII.4 VLP 1987-2009 and 2012. Bound antibodies were visualized by incubation with a second peroxidase labelled antibody followed by a substrate reaction. Pictures were taken with a Leica DMIL LED using a 10× objective at a 100× magnification. See also Example 6.

FIG. 4 shows the ELISA measurement of measles-specific antibodies in cotton rats developed upon immunization with a dose of 1×10e5 TCID50 of MV-Norovirus (see SEQ ID NO:10) as further detailed in Example 7.

FIG. 5 shows the ELISA measurement of Norovirus-specific antibodies in cotton rats developed upon immunization with a dose of 1×10e5 TCID50 of MV-Norovirus (see SEQ ID NO:10). as further detailed in Example 7.

### DEFINITIONS

An “adjuvant” in the field of immunology and as used herein refers to a substance or composition enhancing antigenicity of another substance. This is achieved by specifically stimulating and balancing mainly the Th1, Th2, Th17 and regulatory T (Treg) subsets of T-cells and thus their effector molecules. Usually, immunogenic compositions based on live-attenuated or killed viruses are highly immunogenic per se and thus there might not be the need for an additional adjuvant, whereas an additional adjuvant might still be favourable to balance the provoked immune response. Th1 cells secrete IFN-γ, which activates macrophages and thus induces the production of opsonizing antibodies by B cells. The Th1 response therefore leads mainly to a cell-mediated immunity. Th2 cells mainly secrete cytokines, including IL-4, which induces B cells to make neutralizing antibodies. Th2 cells generally induce a humoral (antibody) response critical in the defense against extracellular pathogens (helminths, extracellular microbes and toxins). Treg cells secrete IL-10 and transforming growth factor (TGF)-β, which modulate helper T-cell activity and suppress some of their functions, inducing tolerance to antigens. Th17 cells as a unique CD4⁺ T-cell subset produce IL-17, IL-17F, IL-6, IL-22 and TNF-α, which, in turn, act on fibroblasts, macrophages, and endothelial and epithelial cells to elicit inflammatory mediator and chemokine release responses.

The term “antigen” as used herein and as commonly used in the field of immunology refers to an “antibody generating” molecule, i.e. a substance, which can elicit an adaptive immune response. An antigen is thus a molecule binding to an antigen-specific receptor, either a T-cell or a B-cell receptor. An antigen is usually a (poly)peptide, but it can also be a polysaccharide or a lipid, possibly combined with a protein or polysaccharide carrier molecule. For the purpose of the various aspects and embodiments of the present invention, the antigen is a (poly)peptide, i.e. an amino acid sequence. In the case of binding to a T-cell receptor, the antigen is presented to the respective T-cell receptor via an antigen-presenting cell as an antigenic peptide bound to a histocompatibility molecule on the surface of the antigen presenting cell, wherein the antigenic peptide has been processed in advance by the antigen presenting cell. Antigen presentation by professional antigen-presenting cells (APC) is the first step towards the initiation of an adaptive immune response carried out by naïve T lymphocytes. Thus, an “antigen” as used herein refers to a molecule, such as a protein or a polypeptide, comprising one or more epitopes that will stimulate a host's immune system to make a humoral and/or cellular antigen-specific response. As such, an antigen can either be located on the surface exposed portion of a recombinant infectious virus or an VLP. An antigen can also be made accessible to the immune system of a subject after processing of a recombinant infectious virus or a VLP or, for certain viruses, another recombinant subviral particle (RSP) and can then be loaded onto a MHC molecule to be presented to a T cell to elicit an immune response, for example.

The terms “attenuation” or “attenuated” as used herein in connection with a virus strain or a material derived therefrom refers to a virus weakened under laboratory conditions which is less vigorous than the respective wild-type virus. An attenuated virus may be used to make a vaccine that is capable of stimulating an immune response and creating immunity, but not of causing illness associated with the wild-type strain.

The term “cDNA” stands for a complementary DNA and refers to a nucleic acid sequence/molecule obtained by reverse transcription from an RNA molecule. As it is a standard method for the person skilled in the art to obtain cDNAs from a given sequence and to further use this cDNA or to clone said cDNA into a vector, preferably a plasmid vector, of interest.

The terms “derived”, “derivative”, “derived from”, “descendant” or “progenitor” as used herein in the context of either a host cell, a cell population or an infectious virus particle according to the present application relates to the descendants of such a host cell or infectious virus particle which results from natural reproductive propagation including sexual and asexual propagation and, in the context of virus nucleic acids, the propagation of the virus genetic material by the host cell machinery. It is well known to the person having skill in the art that said propagation can lead to the introduction of mutations into the genome of an organism resulting from natural phenomena which results in a descendant or progeny, which is genomically different to the parental host cell, however, still belongs to the same genus/species and possesses the same characteristics as the parental host cell. In the context of a nucleic acid sequence or any other biomolecule being “derived from” a certain sequence this term implies that the respective nucleic acid sequence is based on the reference sequence as template, i.e. originating from said sequence, whereas the reference sequence can comprise more sequences, e.g. the whole genome or a full polyprotein encoding sequence, of a virus, whereas the sequence “derived from” the native sequence may only comprise one isolated gene or function fragment thereof, or a coherent fragment thereof. The skilled person can thus easily define a sequence “derived from” a reference sequence in case the reference sequence is known, defined or available, which will, by sequence alignment on DNA or amino acid level, have a high identity to the respective reference sequence and which will have coherent stretches of DNA/amino acids in common with the respective reference sequence (>75% query identity provided that the derived sequence is the query and the reference sequence represents the subject during a sequence alignment). The skilled person can thus clone the respective sequences based on the disclosure provided herein by means of polymerase chain reactions and the like into a suitable vector system of interest, or use a sequence as vector scaffold. The term “derived” is thus not to be understood to define an arbitrary sequence, but a sequence corresponding to a reference sequence it is derived from, whereas certain differences, e.g. certain mutations naturally occurring during replication of a recombinant construct within a host cell, cannot be excluded and are thus comprised by the term “derived from”. Notably, in certain instances, e.g. related to an “antigen” according to the present invention, the term “derived (from)” specifies the nucleic acid sequence the antigen (the antigen being an amino acid sequence) originates from. The skilled person is well aware of the fact that this nucleic acid sequence according to the present invention will then be transcribed and translated in vivo and will be either presented to an immune cell, or it will be further digested and/or processed within a host cell. Therefore, it is more precise to define an antigen in a technical sense based on the corresponding nucleic acid sequence encoding said antigen. In the context of a virus, derivative or descendant or progenitor can thus naturally possess one or more mutations. Such derivatives or descendants resulting from natural phenomena during reproduction or propagation are thus comprised by the term host cell or cell population according to the present disclosure and the skilled person can easily define, by means of molecular biology, microscopy or the like, that the derivative or descendant is indeed derived from a parental host cell of the same genus. These terms, therefore, do not refer to any arbitrary derivative, descendant or progenitor, but rather to a derivative, or descendant or progenitor phylogenetically associated with, i.e. based on, a parent cell or virus or a molecule thereof, whereas this relationship between the derivative, descendant or progenitor and the parent or reference sequence is clearly inferable by a person skilled in the art.

An “epitope” as used herein can be a conformational epitope or a linear epitope. A conformational epitope is a sequence of sub-units, usually amino acids, composing an antigen that comes in direct contact with a receptor of the immune system. An antigen is any substance that the immune system can recognize as foreign. Antigens are usually proteins that are too large to bind as a whole to any receptor so only specific segments, that form the antigen, bind with a specific receptor, such segments usually being called “epitopes”. Likewise, it is only paratope of the respective recognition receptor that comes in contact with the epitope. Whenever a receptor interacts with an undigested antigen, the surface amino acids that come in contact may not be continuous with each other if the protein is unwound. Such discontinuous amino acids that come together in three-dimensional conformation and interact with the receptors paratope are called conformational epitopes. A linear epitope linear or a sequential epitope, in contrast, is an epitope that is recognized by the corresponding antigen receptor by its linear sequence of amino acids, or primary structure.

The terms “fragment” or “functional fragment” as used herein in the context of amino acid/protein, or nucleic acid sequences, including sequences of viruses and vectors and scaffold based on said viruses, refers to a at least one coherent fragment, which is comprised by a whole, the reference sequence. The terms thus mean a portion or fragment of a whole capable of performing, in whole or in part, a function of the whole. For example, a biologically functional portion of a molecule means a portion of the molecule that performs a biological function of the whole or intact molecule, i.e. a nucleic acid sequence or an amino acid sequence, preferably an antigen sequence, according to the present invention. For example, a functional portion of a nucleic acid sequence or the encoded protein is a fragment or portion of the specified nucleic acid sequence or the encoded protein that comprises at least one coherent part of the native or wild-type sequence. Functional fragments are thus suitable for vaccination purposes to focus on a sub-fragment of a whole protein to specifically elect those parts of immunogenic interest. As such, a “functional fragment” per se may have 100% sequence identity to the respective stretch of the sequence it is derived from (the reference sequence) over a given length of alignment. Still, a single nucleotide or amino acid may not be unambiguously attributable to a parent structure. In terms of absolute sequence identity, a “functional fragment” according to the present invention will thus not only be 100% identical to a given length over an alignment with the parent structure, but the “functional fragment” will have at least 15, at least 20, at least 25, at least 30, at least 35 or at least 40 nucleic acid positions in common with the respective parent structure (on DNA and/or amino acid level) it is derived from. In case that the parent sequence will comprise at least one mutation, the “functional fragment” can also comprise said mutation(s), or the “functional fragment” can comprise at least one additional mutation to optimize its properties (e.g., expression rate, folding characteristics, presentation to immune cells, immunogenicity in vitro and in vivo) desirable according to the present disclosure. The fragment will, however, usually be shorter than the sequence it is derived from.

The term “host cell” or “host cell population” as used herein refers to at least one but preferably more than one host cell(s). The term host cell comprises non-recombinant cells, i.e. cells that were not immortalized or transformed or manipulated in a purposive manner. Including also primary cells. To be suitable for certain aspects of the present invention, the host cell(s) of the cell population must be able to support the measles virus replication cycle, i.e. the cell(s) must be susceptible to measles virus or recombinant measles virus infection and the cell(s) must suitable for the subsequent propagation or replication cycle, including replication, translation encapsidation of the RNA of the virus and budding from the host cell to be released as virus particle. Several eukaryotic host cells are fulfilling this purpose are cited herein or known to the skilled person.

An “immunogenic composition” as used herein refers to a composition which is able to induce any immune response in a subject or a cellular system in vitro. A vaccine or vaccine composition per se is thus also comprised by the term immunogenic composition. However, it is well known to the skilled person that for being suitable to administration to an animal, an immunogenic composition can additionally comprise suitable pharmaceutically and/or veterinary acceptable carriers. The term “vaccine composition” as used herein refers to composition, preferably based on a virus particle and/or a VLP, which is able to induce a protective immune response in a subject.

As used herein, the term “immune response” refers to an innate or adaptive immune response, e.g. the T cell response or to the increased serum levels of antibodies as resulting from exposure to an antigen due to a B-cell response, or presence of neutralizing antibodies to an antigen, or it refers to an innate immune response, e.g. mediated by the complement system and cells of the innate immune system like macrophages or dendritic cells. It also refers to an allergic response, inter alia including mast-cell, eosinophil or NK-cell action, T-cells, B-cell secreted antibodies. The term immune response is to be understood as including a humoral response and/or a cellular response and/or an inflammatory response.

The term “measles virus” (MV) or “measles virus particle” as used herein, is to be understood as referring to any particle comprising or derived from MV including native (wild-type) or recombinant MV, optionally further comprising artificial elements or mutations, preferably purposive mutations, either in live, attenuated, inactivated or killed form. Also covered by said term is a virion and a virus-like particle derived from, and thus comprising structural elements of, MV. The term “measles virus backbone” is used interchangeably with the term “measles virus scaffold” herein and (MV being a negative-sense single-stranded RNA virus of the Family Paramyxoviridae) refers to the antigenomic (+) RNA strand of a measles virus, usually provided as cDNA, which may encode an infectious MV, but which may also be used as expression vector or expression construct for sequences encoding non-MV antigens or sequences.

The term “modified” in the context of a nucleic acid or amino acid sequence comprises any recombinant, chemical or enzymatic modification of the protein, peptide, polypeptide and enzyme or of the nucleic acid sequence encoding the same.

A “monovalent” vaccine is meant to refer to a vaccine designed to immunize against a single antigen(s) or single microorganism (including viruses), whereas “polyvalent” or “multivalent” vaccines aim to immunize against several strains (or genotypes) of the same microorganism (including viruses), or against several microorganisms (including viruses). A “bivalent” as used herein refers to a vaccine aiming at immunizing against two different strains (or genotypes) of the same microorganism (including viruses), or against two different microorganisms (including viruses).

As used herein, the term “pharmaceutically acceptable” refers to those compounds, materials, compositions, and/or dosage forms which are, within the scope of medical judgment or within the definition of any regulatory medical authority, suitable for contact with the cells, tissues, or components of a subject, i.e. human beings and animals, including contact with malignant cells or tissues of a subject, without excessive toxicity, irritation, allergic response, or other complications or side-effects commensurate with a reasonable benefit/risk ratio for a subject/patient.

The term “protection” or “protective immunity” or “protective immune response” as used herein refers to the ability of a substance to induce the production of serum antibodies (adaptive humoral response) and/or a cellular response during immunization to protect (partially or totally) a subject having been treated with the substance against a virus of interest during a subsequent infection or vaccination. Thus, an animal showing a protective immune response will show limited growth and spread if infected with the respective naturally occurring virus afterwards. Also an induced innate immune response mediated by immune cells of the innate immune system like macrophages or dendritic cells, can contribute to the protective effect of a substance, in case the substance stimulates said innate immune response, which can in turn contribute to enhance an adaptive immune response.

The term “reactogenicity” refers to the property of a vaccine of being able to produce common, “expected” adverse reactions, especially excessive immunological responses and associated signs and symptoms like fever, sore arm at injection site, etc.

The terms “recombinant”, “genetically modified” or “genetical engineering”/“genetically engineered” as used herein refer to any nucleic acid molecule and/or an amino acid molecule or a host cell implying a targeted and purposive manipulation and/or modification achieved by means of molecular biology or protein engineering, e.g. by introducing a heterologous sequence into another host cell, by modifying a naturally occurring nucleic acid sequences and the like. Further modifications include, but are not limited to, one or more point mutation(s), one or more point mutation(s), e.g. for targeted protein engineering or for codon optimization, deletion(s), and one or more insertion(s) of at least one nucleic acid or amino acid molecule, modification of an amino acid sequence, or a combination thereof. The terms can also imply a sequence, which per se occurs in nature, but has been purposively treated by means of molecular biology isolated from its natural environment in vitro so that the sequence represents an artificial sequence.

The term “regulatory sequence” as used herein refers to a nucleic acid sequence which can directly influence, or which can indirectly be influenced (e.g. by binding to a transcription factor), to effect the transcription and/or translation of a target nucleic acid sequence of interest. The term thus refers to promoter and terminator sequences or to polyadenylation signals, a Kozak sequence and the like.

“Subject”, as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In an embodiment, the subject is a human being.

“Treat”, “treating” and “treatment”, as used herein, mean the treatment of a disease in a mammal, e.g. in a human, including (a) inhibiting the disease, i.e. arresting its development; (b) relieving the disease, i.e. causing regression of the disease state; and (c) curing the disease. The terms “prevent” or “preventing” imply that a prophylactic treatment is provided before the onset of the disease or before the onset of the symptoms associated with a disease to be prevented.

The term “vector”, “vector backbone”, “(vector) scaffold” or “plasmid vector” or “expression vector” as used herein defines a system comprising at least one vector suitable for transformation, transfection or transduction of a host cell. The vector is a suitable cargo for cloning to insert sequences of interests, e.g. antigenic sequences, which have to be positioned at the appropriate cloning sites. A vector per se thus denotes a cargo for the delivery of a biomolecule into a host cell of interest, wherein the biomolecule includes a nucleic acid molecule, including DNA, RNA and cDNA, or, in the case of a transfection system as vector, an amino acid molecule, or a combination thereof. A preferred vector according to the present invention is a plasmid or expression vector, which can be produced in a host cell. An expression vector can comprise one vector encoding at least one target molecule, preferably a nucleic acid molecule, to be introduced into a host cell. A vector of the vector system can also comprise more than one target molecules to be introduced. Alternatively, the vector system can be built from several individual vectors carrying at least one target molecule to be introduced. An expression vector additionally comprises all elements necessary for driving transcription and/or translation of a sequence of interest in a host cell, the expression vector is designed for. These elements comprise, inter alia, regulatory elements, which are involved in the regulation of transcription, including promoters and the like functional in the host cell of interest. Furthermore, an expression vector comprises an origin of replication and optionally depending on the type of vector and the intended use a selectable marker gene, a multiple cloning site, a tag to be attached to a sequence of interest, a chromosomal integration cassette and the like. The choice and possible modification of a suitable expression vector for use with a respective host cell and sequence of interest to be inserted into the expression vector is well within the capabilities of the person skilled in the art.

A “viral particle” or “virus particle” or “recombinant and/or infectious virus particle” as used herein refers to a single particle derived from a viral nucleic acid, which is located outside a cell. The viral particle thus represents the mature and infectious form of a virus. As the viral particle contains genetic information, it is able to replicate and/or it can be propagated in a susceptible host cell. Depending on the complexity of a virus, the viral particle comprises nucleic acid and polypeptide sequences and, optionally lipids, preferably in the form of a lipid membrane derived from the host cell. The virus particle may encode a sequence which can form a virus-like particle.

A “virion” as used herein refers to a single particle derived from a nucleic acid encoding a virus or a viral particle, which is located outside a cell containing nucleic acids and thus being able to replicate or to be transcribed in a suitable host cell.

A “virus-like particle” or “VLP” as used herein refers to at least one virus particle, which does not contain any nucleic acid. VLPs or VLPs usually comprise at least one viral structural protein from a virus they are derived from and optionally lipid constituents. As such, they can be used to for vaccination or for inducing an immunogenic reaction in a subject, due to the absence of nucleic acids they will, however, not be able to replicate in a host cell and are thus non-replicative. VLPs are thus understood to represent particles lacking genetic information and are thus non-replicative. VLPs per se are thus non-infectious in the sense that they cannot replicate in a cell to give rise to new viral particles and thus to spread to further cells after a replicative cycle. Still, VLPs, after their assembly and based on the molecules exposed on their surface, can interact with a host cell and/or host cell molecules, e.g. surface receptors, or, after uptake and/or processing by an immune cell, e.g. an antigen-representing cell, epitopes or antigens comprised by a VLP can be presented or cross-presented by the immune cell to effector cells. By means of this interaction, VLPs can induce an immune response in an organism. This ability makes VLPs suitable structures for the provision of immunogenic or vaccine compositions.

A “virus sample”, “virus material” or the like refers to a material comprising at least one of a (recombinant infectious) virus particle and/or a virion and/or a VLP.

Whenever the present disclosure relates to the percentage of the homology or identity of nucleic acid or amino acid sequences to each other, these values define those as obtained by using the EMBOSS Water Pairwise Sequence Alignments (nucleotide) programme (http://www.ebi.ac.uk/Tools/psa/emboss_water/nucleotide.html) nucleic acids or the EMBOSS Water Pairwise Sequence Alignments (protein) programme (http://www.ebi.ac.uk/Tools/psa/emboss_water/) for amino acid sequences. Those tools provided by the European Molecular Biology Laboratory (EMBL) European Bioinformatics Institute (EBI) for local sequence alignments use a modified Smith-Waterman algorithm (see http://www.ebi.ac.uk/Tools/psa/and Smith, T. F. & Waterman, M. S. “Identification of common molecular subsequences” Journal of Molecular Biology, 1981 147 (1):195-197). When conducting an alignment, the default parameters defined by the EMBL-EBI are used. Those parameters are (i) for amino acid sequences: Matrix=BLOSUM62, gap open penalty=10 and gap extend penalty=0.5 or (ii) for nucleic acid sequences: Matrix=DNAfull, gap open penalty=10 and gap extend penalty=0.5.

### DETAILED DESCRIPTION

Human Noroviruses are the leading cause of foodborne illnesses across all age groups and meanwhile account for nearly one fifth of all acute gastroenteritis infections on a global level. Human Noroviruses are presently superseding rotavirus as the most common cause of acute gastroenteritis amongst children, in particular in regions where rotavirus vaccination has been widely and successfully deployed.

As detailed above, there is presently no approved Norovirus vaccine despite the emerging global relevance of Norovirus infections, in particular in children or in the elderly or immunosuppressed population. The inventors thus established a vaccine candidate with high immunogenicity by providing an expression construct allowing the generation of stable and immunogenic VLPs. In addition, the relevant Norovirus antigens were expressed by a self-replicating and therefore self-adjuvanting vector system which may be used to increase the potency of the vaccine candidate. The strategy of co-expression disclosed herein was reported for mono and bi-/multivalent vaccine candidates and can thus also be used for the development of new (mono- or multivalent) Norovirus vaccine candidates in a rapid manner to address the urgent need of promptly generating new and potent vaccine candidates facing the rapid emergence of new and epidemiologically important Norovirus genogroups and genotypes.

In a first aspect, there may this be provided an immunogenic composition comprising at least one recombinant Norovirus antigen, and/or a sequence encoding the same, wherein the at least one recombinant Norovirus antigen is encoded by at least one nucleic acid sequence encoding at least one Norovirus VP1 sequence and at least one Norovirus VP2 sequence, wherein the at least one nucleic acid sequence is operably linked to a vector backbone, wherein the immunogenic composition optionally comprises at least one pharmaceutically acceptable carrier and/or excipient.

In one embodiment, the Norovirus VP1 sequence and/or the Norovirus VP2 sequence may be independently derived from a Norovirus of the GII genogroup, preferably from a Norovirus having a GII.4 genotype, preferably wherein the Norovirus VP1 sequence and the Norovirus VP2 sequence may be encoded by the same nucleic acid sequence and optionally may be separated by at least one linker sequence.

The term “linker” as used herein refers to any coding or non-coding nucleic acid sequence. In one embodiment, the linker sequence may be a regulatory sequence, for example, comprising at least one terminator and/or promoter sequence allowing the separation of a sequence encoding at least one VP1 and/or VP2 sequence from the same or a different genotype/genogroups. Relying on a linker sequence allows the creation of monovalent and multivalent vaccines carrying more than one VP1 and/or VP2 sequence to obtain a vaccine candidate with maximum coverage of prevailing Norovirus antigens. Representative linker sequences are comprised in SEQ ID NOs:9 and 10 and are shown in SEQ ID NOs:17 and 18 (ATUs used as linker regions to insert VP1 and/or VP2 sequences in a targeted way to allow both measles virus replication as well as expression of VP1 and/or VP2 in a functional manner so that VLPs can be formed from the Norovirus sequences inserted, measles regulatory terminator and/or promoter regions).

In another embodiment, the linker sequence may encode a protein sequence which serves as flexible linker connecting VP1 and/or VP2 antigen sequences as long as the linker enhances VLP formation, e.g. the linker may encode a short loop or helical segment further enhancing VLP formation. In certain embodiments, the linker may be a 2A self-cleaving peptide (cf. Liu et al., Sci. Rep., 2017; 7:2193).

The linker sequence may be an artificial sequence. In certain embodiments, the linker sequence may be derived from a sequence of a virus belonging to the family Paramyxoviridae, for example, from measles virus, respiratory syncytical virus (RSV), or Vesicular stomatitis virus (VSV), the latter from the family Rhabdoviridae. The linker sequence does not need to be endogenous to the genome of a virus used as vector backbone in modified form.

The linker may thus fulfil the function of a spacer either to separate two sequences encoding antigens from each other, or the linker may have regulatory functions, i.e. by encoding a regulatory sequence acting in cis or in trans as regulators. The linker may further include a marker sequence (DNA or encoding a marker protein) to visualize expression, or the linker may comprise elements to allow proper function of the backbone used as vector.

The above immunogenic composition allows both, the provision of an infectious replicative particle which is self-adjuvanting and can be used to propagate itself and thus an immune response in a subject, as well as the provision of non-enveloped VLPs as derived from Norovirus capsid proteins. If desired, the different populations can be further separated from each other as further detailed below.

In one embodiment according to the various aspects disclosed herein, wherein the VP1 and/or the VP2 sequence are independently selected from a Norovirus having a GII.4 genotype, preferably from a sequence from a Norovirus Hu/GII.4/Sydney/NSW0514/2012/AU, a Norovirus Hu/GII.4/Orange/NSW001P/2008/AU, a Norovirus Hu/GII.4/NIHIC17.5/2012/USA, a Norovirus Hu/GII.4/G114-NG1242/2011/JP, or a Norovirus Hu/GII.4/Hiroshima4/2012/JP, wherein the VP1 and the VP2 sequence are derived from the same Norovirus, or wherein the VP1 and the VP2 sequence are derived from a different Norovirus, optionally wherein the VP1 and the VP2 sequence are modified in comparison to the reference sequence. The term “modified” or “modification” in this context is to be understood as at least one targeted mutation of a (c)DNA or RNA sequence to optimize a reference sequence as retrieved from a Norovirus to obtain, for example, more stable sequences better forming VLPs, codon-optimized sequences showing a better transcription, translation and assembly, or for obtaining a consensus sequence showing a broader scope of protection against various Norovirus genogroups/genotypes and the like.

Notably, Norovirus sequences are readily retrievable from public databases and can be chemically synthesized, optionally including codon optimization, for cloning purposes and vaccine design. Under clinical settings, a Norovirus GI and GII duplex RT-PCR can be performed targeting the 5′ end of capsid gene for norovirus genotyping along with a norovirus GI or GII RT-PCR targeting the ORF1/ORF2 overlap for the identification of potential recombinants in patients.

Hu/GII.4/Sydney/NSW0514/2012/AU (*) (GenBank Entry: JX459908) first identified in Sydney in 2012 was chosen as one of the candidates studied in view of the fact that this GII.4 genotype of Norovirus caused severe and pandemic Norovirus infections and was dominating over other genotypes. For the past two decades, Norovirus pandemic variants have emerged around every 3-5 years, and have dominated until they are replaced by alternate strains. However, this scenario changed in 2016 with the co-circulation of six prevalent viruses, three of which possessed the pandemic GII.4 Sydney 2012 capsid for which reason this Norovirus was selected as source for a vaccine candidate. In 2016, there was a decline of the pandemic Sydney 2012 variant, concomitant with the emergence of two novel GII.4 recombinant viruses, both of which retained the Sydney 2012 capsid but acquired new non-structural regions (GII.P4 New Orleans 2009/GII.4 Sydney 2012 and GII.P16/GII.4 Sydney 2012; see Lun et al., Emerging recombinant noroviruses identified by clinical and waste water screening. Emerg. Microbes Infect. 2018; 7:50). Therefore, it was followed that the VP1 and VP2 sequences of the original virus seem to be rather conserved. It was thus also speculated that the Hu/GII.4/Sydney/NSW0514/2012/AU sequences could serve as one of at least two sequences to obtain a multifunctional consensus sequence to obtain a vaccine providing broad and more than seasonal protection against Norovirus infection. Based on the prototypic expression construct, further mono- and multivalent Norovirus vaccine candidates were cloned to show that the strategy disclosed herein may be rapidly adapted to new emerging Norovirus strains, as well as known genogroups and genotypes (e.g. Norwalk virus belonging to the GI genogroup) and can be used to provide broad protection against different genogroups/genotypes, which becomes of increasing importance in a global society where different Norovirus strains are dominant in certain regions due to climatic and other reasons.

Other Norovirus strains can be selected from, but are not restricted to Norovirus Hu/GII.4/NIHIC17.5/2012/USA (GenBank Entry: KF429790), Norovirus Hu/GII.4/Orange/NSW001P/2008/AU (*) (GenBank Entry: GQ845367), Norovirus Hu/GII.4/G114-NG1242/2011/JP (GenBank Entry: AB972502.1), or a Norovirus Hu/GII.4/Hiroshima4/2012/JP (GenBank Entry: AB972494.1). Further GII.4 variants can be selected from CHDC5191/1974/US (GenBank Entry: FJ537134), Lorsdale/1993/GB (GenBank Entry: X86557), Camberwell/1994/AU (GenBank Entry: AF145896), Hu/NLV/Dresden174/pUS-Nor11/1997/GE (*) (GenBank Entry: AY741811), Hu/Houston/TCH186/2002/US (GenBank Entry: EU310927), Hu/GII.4/Kaiso/030556/2003/JP (GenBank Entry: AB294779), Hu/NoV/Farmington Hills/2002/USA (*) (GenBank Entry: AY502023.1), Norovirus Hu/NLV/Dresden174/pUS-Nor11/1997/GE (GenBank Entry: AY741811.1), Norovirus Hu/GII.4/Hunter504D/040/AU (*) (GenBank Entry: DQ078814.2), Norovirus Hu/GII.4/Shellharbour/NSW696T/2006/AUS (*) (GenBank Entry: EF684915.2), Norovirus Hu/GII.4/Osaka1/2007/JP (GenBank Entry: AB541319.1), Norovirus Hu/GII.4/Sutherland/NSW505G/2007/AUS (GenBank Entry: (GenBank Entry: GQ845368.2), or a Norovirus Hu/GII.4/JB-15/KOR/2008 (GenBank Entry: HQ009513.1). GII.4 variants marked with an asterisk (*) were associated with a pandemic of acute gastroenteritis and demonstrated a global distribution (Eden et al., Recombination within the pandemic norovirus GII.4 lineage, J. Virol. 2013 June; 87(11):6270-82. doi: 10.1128/JVI.03464-12).

Further suitable Norovirus sequences, including non-GII.4 sequences recently of interest in Asia, are disclosed in Chi-Wai et al. (Bimodal Seasonality and Alternating Predominance of Norovirus GII.4 and Non-GII.4, Hong Kong, China, 2014-2017, CDC EID Journal, vol. 24, 2018). Another Norovirus, prototypic, but not limiting for a genogroup GI strain, is Norwalk virus (Hu/GII.6/CHDC4073/1984/USA, GenBank entry: JX846927.1).

Selecting a Norovirus genotype having a pandemic potential may be preferable in certain embodiments. In view of the rapid antigenic drift and shift of Norovirus and differences in global Norovirus genotype distribution, it may be preferable to provide an immunogenic composition comprising at least to different antigens (e.g. VP1 and VP2) being able to form a functional, i.e. a self-assembling and immunogenic antigen presenting, VLP. In another embodiment, it may be preferable to combine different VP1 sequences from different genogroups/genotypes VP1 being able to self-assemble into a VLP.

In certain embodiments, the at least one sequence encoding at least one VP1 and/or VP2 antigen may be modified in comparison to the wild-type sequence as retrievable from a wild-type isolate as sequenced from material obtained from a patient.

In other embodiments, it may be preferable to design a bi- or multivalent vaccine comprising at least one VP1 or VP2 sequence from a first genogroups or genotype and further comprising another at least one VP1 or VP2 sequence from another genogroups or genotype.

In certain embodiments according to the various aspects disclosed herein, an immunogenic composition may comprise more than one distinct Norovirus VLP, for example by creating a tandem construct wherein the vector backbone comprises a sequence encoding a first VP1 and optionally a second VP2 sequence building one first VLP, and wherein the vector backbone further comprises a third and optionally a fourth sequence encoding a different VP1 and optionally a different VP2 sequence. These sequences can be separated by adequate linker sequences as disclosed herein and they can be cloned into the same, or into a different ATU sequence in the vector backbone. The immunogenic composition thus created will thus comprise antigenic diverse VLPs derived from different Norovirus isolates. This can provide a multiplication of the immunogenic function and in particular a wider scope of protection against different Norovirus genotypes so that an individual can be protected against infection with different Norovirus genogroups or genotypes emerging from different regions worldwide.

In certain embodiments according to the various aspects of the present invention it may be preferable to use a VP1 sequence together with a VP2 sequence to obtain more stable VLPs showing a better antigen presentation to the immune system.

Depending on the at least one VP1 antigen chosen, and in turn the sequences encoding the same, there may also be provided an immunogenic composition comprising at least one recombinant Norovirus antigen, wherein the antigen is encoded by at least one nucleic acid sequence encoding at least one Norovirus VP1 sequence, wherein the at least one nucleic acid sequence is operably linked to a vector backbone, wherein the immunogenic composition optionally comprises at least one pharmaceutically acceptable carrier and/or excipient. In this embodiment, the use of a suitable linker allows the provision of at least two, at least three, at least four, at least five, at least six, at least seven, at least eight, at least 9 or even at least ten different VP1 sequences all provided in the same vector backbone so that an immunogenic composition can be provided that comprises at least two different VP1 Norovirus antigens, preferably obtained from different Norovirus genogroups or genotypes so that a bi- or multivalent vaccine can be provided that simultaneously provides a subject protection against two or even several different Noroviruses.

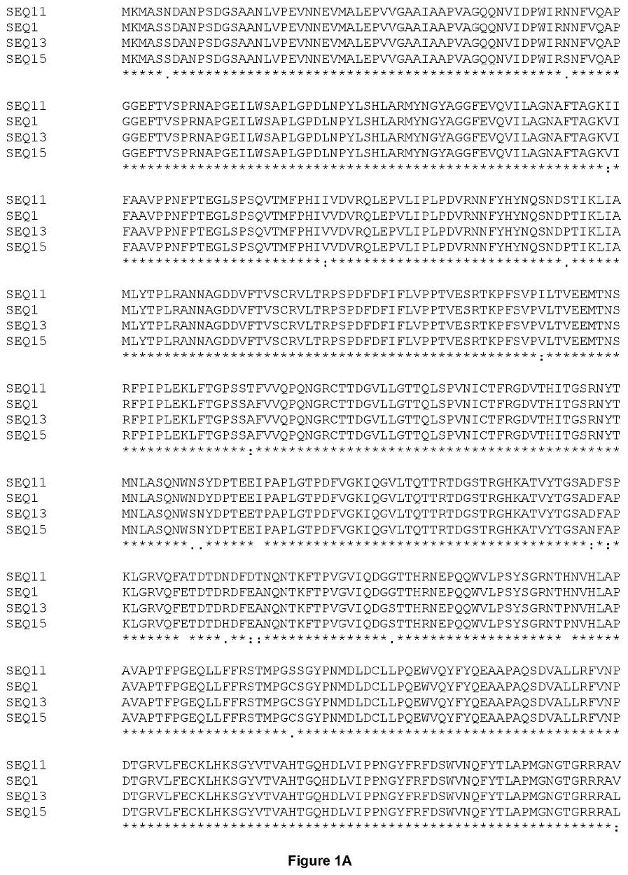

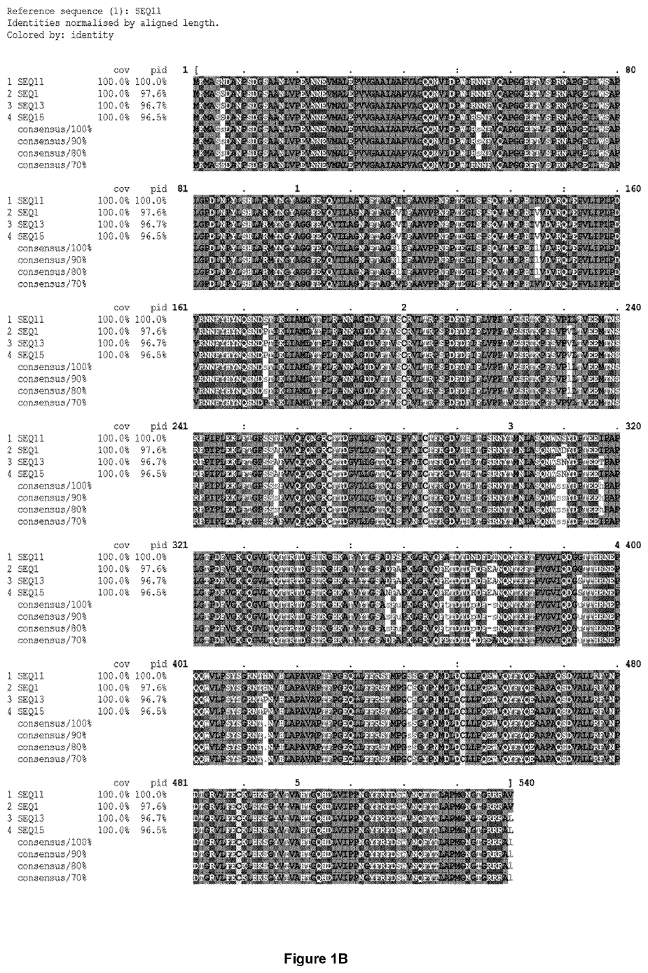

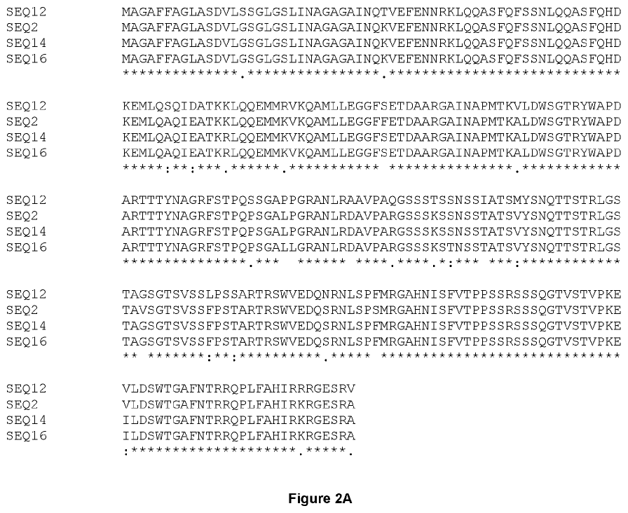

In certain embodiments, the at least one VP1 and/or the at least one VP2 sequence represent(s) a conserved consensus sequence, preferably a consensus sequence within the same genotype, i.e. GII.4, GII.17 and the like. As detailed above, Norovirus strains show a regional distribution and related, but not identical, Norviruses may be dominating in certain regions. Therefore, it may be suitable to identify a consensus sequence as detailed in FIG. 1 and FIG. 2 to obtain an optimum protection of a vaccine candidate comprising antigenic sequences covering different genotypes. The skilled person can easily define a suitable consensus sequence based on at least two, preferably at least three sequences compared as depicted for the disclosure provided with FIG. 1 and FIG. 2. Highly conserved amino acid positions will always be present in a consensus sequence. In case sequence comparison reveals that certain amino acid positions are less conserved over the same genotype, more than one VP1 and/or VP2 sequence encoding the respective capsid proteins and thus antigens from a Norovirus may be selected. The consensus sequence is determined over the whole length of a sequence encoding a Norovirus VP1 and VP2 sequence. The consensus sequence may comprise mutations and/or the consensus sequence may be truncated. Preferably, the consensus sequence has at least 90%, 91%, 92%, 93%, 94%, 95%, 96%, 97%, 98% or 99% amino acid sequence identity to each reference sequence used for creating the consensus sequence over the whole length of the respective reference amino acid sequences.

In certain embodiments, the at least one recombinant Norovirus VP1 antigen may be selected from an amino acid sequence of SEQ ID NOs:1, 11, 13 or 15 or a nucleic acid sequence encoding the same (e.g., SEQ ID NOs:3, 5) or an amino acid sequence having at least 90%, 91%, 92%, 93%, 94%, 95%, 96%, 97%, 98% or 99% sequence identity thereto, and wherein the at least one recombinant Norovirus VP2 antigen is selected from an amino acid sequence of SEQ ID NOs:2, 12, 14 or 16 or a nucleic acid sequence encoding the same (e.g., SEQ ID NOs:4, 6), or an amino acid sequence having at least 90%, 91%, 92%, 93%, 94%, 95%, 96%, 97%, 98% or 99% sequence identity thereto. Any other Norovirus VP1 and/or VP2 sequence, in particular a seasonal or recent sequence emerging and having been identified and thus being publicly available, may be used. SEQ ID NO:1 represents the VP1 amino acid sequence from Hu/GII.4/Sydney/NSW0514/2012/AU and SEQ ID NO:3 represents the corresponding nucleic acid sequence. SEQ ID NO:2 represents the VP2 amino acid sequence from Hu/GII.4/Sydney/NSW0514/2012/AU and SEQ ID NO:4 represents the corresponding nucleic acid sequence. SEQ ID NOs:5 and 6 represent codon-optimized nucleic acid sequences of VP1 and VP2 of Hu/GII.4/Sydney/NSW0514/2012/AU, respectively. SEQ ID NO:7 then shows an artificial VP1-linker-VP2 constructs derived from of Hu/GII.4/Sydney/NSW0514/2012/AU and SEQ ID NO:8 shows a codon-optimized VP12-linker-VP2 construct as derived from of Hu/GII.4/Sydney/NSW0514/2012/AU sequences as reference sequences for VP1 and VP2. SEQ ID NOs:11 and 12 show the amino acid sequence of VP1 and VP2 of Norovirus Hu/GII.4/NIHIC17.5/2012/USA, respectively. SEQ ID NOs:13 and 14 show the amino acid sequence of VP1 and VP2 of Norovirus Hu/GII.4/G114-NG1242/2011/JP, respectively. SEQ ID NOs:15 and 16 show the amino acid sequence of VP1 and VP2 of Norovirus Hu/GII.4/Hiroshima4/2012/JP, respectively. These sequences are represented here in an exemplary manner to provide some more recent Norovirus GII.4 sequences. As detailed above, the skilled person can easily retrieve further Norovirus sequences of different genogroups and genotypes from publicly available databases.

According to the various embodiments of the present invention, the vector backbone serving as platform to introduce at least one, preferably at least one VP1 and at least one VP2 Norovirus antigen according to the present invention may be selected from the group consisting of a measles virus backbone, a lentivirus backbone, a Vesicular stomatitis virus (VSV) backbone, or a vaccinia Ankara backbone, preferably wherein the vector backbone may be from a recombinant live-attenuated measles virus strain, the measles virus strain preferably being selected from the group consisting of a Schwarz strain, a Zagreb strain, an AIK-C strain and a Moraten strain. Preferably, a vector backbone is a modified recombinant backbone to fulfill the needs of a safe vaccine as well as the needs of a suitable multipurpose cloning vector. Preferably, the backbone should be recombinantly optimized to fulfill the needs for a viral vaccine vector.

A preferred vector backbone according to the present disclosure is a measles virus (MV) backbone. For example, EP 1 939 214 B1 discloses that live attenuated RNA viruses especially the measles vaccine, has been used in hundreds of millions of children and has been proven to be effective and safe. This vaccine induces life-long immunity after one or two injections. It is easily produced on a large scale at low cost in most countries. These advantages make measles virus, especially attenuated vaccine strains, a good candidate vector to immunize children. Measles virus (MV) belongs to the genus Morbillivirus in the family Paramyxoviridae. It is an enveloped virus with a non-segmented RNA genome of negative polarity (15,894 bp). Measles can only be contracted once as the immune system mounts a strong specific response and establishes life-long memory protecting against re-infection. Such protection is based on both the production of antibodies and memory cytotoxic CD8+T lymphocytes (CTL). Pathogenic strains strongly disrupt haematopoiesis (Ameborn et al., 1983; Kim et al., 2002; Okada et al., 2000) thus resulting in transitory immunosuppression responsible for most deaths due to measles infection in developing countries. In contrast to primary strains, attenuated strains do not induce immunosuppression (Okada et al., 2001). Transcription and replication of measles virus does not involve the nucleus of the infected cells but rather take place in the cytoplasm of said infected cells. The genome of the measles virus comprises genes encoding six major structural proteins from the six genes (designated N, P, M, F, H and L) and two additional non-structural proteins from the P gene. The gene order is the following: 3′, N, P (including C and V), M, F, H, and L large polymerase protein at the 5′ end. The genome further comprises non coding regions in the intergenic region M/F; this non-coding region contains approximately 1000 nucleotides of untranslated RNA. The cited genes respectively encode the leader peptide (I gene), the proteins of the nucleocapsid of the virus, i.e., the nucleoprotein (N), the phosphoprotein (P), and the large protein (L) which assemble around the genome RNA to provide the nucleocapsid. The other genes encode the proteins of viral envelope including the hemagglutinin (H), the fusion (F) and the matrix (M) proteins. The Edmonston strain of measles virus was already isolated in 1954 by culture in primary human cells (Enders et al., 1954). Adaptation to chicken embryonic fibroblasts produced vaccine seeds that were furthermore attenuated by subsequent passages in chicken embryonic fibroblasts (Schwarz et al., 1962). The Schwarz and Moraten strains that possess identical nucleotide sequences (Parks et al., 2001a; Parks et al., 2001 b) constitute the most frequently used measles vaccine. Vaccination with one or two injections induces life-long immunity (Griffin et al., 2001; Hilleman et al., 2002). The inventors of EP 1 939 214 B1 have developed a vector using the Schwarz MV, the most commonly used measles vaccine in the world (Combredet et al., 2003). This vector can stably express a variety of genes or combination of large genes for more than 12 passages.

Recombinant MV vectors containing 4,000-5,000 additional nucleotides were produced, representing an additional 30% of genome. These viruses were produced in cell culture at titers comparable to standard MV. To optimize the output of the reverse genetics system, the antigenomic viral cDNA was placed under the control of the T7 phage RNA polymerase promoter with an additional GGG motif required for optimal efficacy. To allow exact cleavage of the viral RNA, a hammerhead ribozyme was inserted between the GGG motif and the first viral nucleotide, and the ribozyme from hepatitis delta virus was placed downstream of the last viral nucleotide. The resulting pTM-MVSchw plasmid enabled the production of the corresponding virus using a previously described reverse genetics system based on the transfection of human helper cells (Radecke et al., 1995). Furthermore, EP 1 939 214 B1 discloses that pTM-MVSchw plasmid was modified for the expression of foreign genes by the introduction of additional transcriptional units (ATU) at different positions of the genome. These ATUs are multi-cloning site cassettes inserted in a copy of the intergenic N-P region of the viral genome (containing the cis acting sequences required for transcription). With the help of the ATUs, and following precise cloning rule, chimeric viruses can thus be created using MV as backbone for the expression of a non-MV sequence.

To be suitable as general purpose expression vector, MV thus has to be attenuated and has to fulfil certain characteristics of a classical cloning vector, i.e. suitable unique cloning sites have to be present. Furthermore, the so-called “rule of six” has to be fulfilled if working with a sequence encoding an infectious replicative MV. This rule implies the total number of nucleotides present in the (antigenomic) cDNA encoding MV amounts to a multiple of six, which allows sufficient replication of genome RNA of the measles virus.

In certain embodiments, a suitable Kozak sequence may be included before the start codon for a sequence encoding a VP1 and/or a VP2 protein according to the present disclosure. A Kozak sequence is a sequence which occurs on eukaryotic mRNA and usually has the consensus (gcc)gccRccAUGG. The Kozak consensus sequence plays a major role in the initiation of the translation process and may vary from species to species. The skilled person can easily define a suitable Kozak sequence. Examples in the context of the present invention can be derived from SEQ ID NOs:9 and 10, where a Kozak sequence precedes the coding sequence of the VP1 and/or the VP2 sequence.

Furthermore, unique cloning sites have to be present to allow the insertion of a heterologous sequence of interest to generate a chimeric MV including an insert of interest. Suitable, but not limiting, restriction enzyme cutting sites can be selected from a BsiWI (5′ cloning site for heterologous gene) and a BssHII (3′ cloning site for heterologous gene) site as also present in SEQ ID NOs:9 and 10.

In a preferred embodiment according to the various aspects of the present disclosure, a MV backbone may comprise at least one ATU. An ATU, or several ATUs, may, for example, be inserted between the regions encoding MV P and M proteins, or between the regions encoding H and L proteins. Suitable ATUs according to the present disclosure are shown in SEQ ID Nos:17 and 18, which represent the 5′ and 3′ ATU as present in SEQ ID NO:10 and represent measles terminator/promotor regions for heterologous gene expression, i.e. for the expression of a sequence encoding VP1 (SEQ ID NO:9) or VP1 and VP2 (SEQ ID NO:10). As detailed above, additional ATUs can be inserted between coding regions of MV genes provided that the insertion does not to disrupt the biological activity of MV (transcription, expression and self-assembly in a host cell).

This basis recombinant and infectious measles vector thus allows the design of combined vaccines based on a live attenuated vaccine strain so that the recombinant measles virus vector may be used as a versatile backbone or scaffold for the introduction, production and purification of infectious virus particles or as cargo for the expression of VLPs consisting essentially of viral structural proteins or epitopes other than those derived from a measles virus strain.

In particular, in the context of combating viruses having a high antigenic drift and/or shift tendency, as it is the case for Norovirus or Influenza, the recombinant and specifically modified MV backbone can thus be used as platform to rapidly establish seasonal vaccines rapidly to fulfil the need of providing a new vaccine generation at short notice.

In certain embodiments, the vector backbone may comprise a nucleotide sequence of the full-length, infectious antigenomic (+) RNA strand of a measles virus, preferably wherein the immunogenic composition comprises a sequence of SEQ ID NO:10, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto.

In another embodiment, the full-length, infectious antigenomic (+) RNA strand of a measles virus may be further modified to comprise suitable mutations or truncations, for example a full or partial knock-out of a structural or non-structural gene, or a mutation having a further attenuating effect, or a targeted mutation optimizing the replication rate and the like.

In yet a further embodiment, the immunogenic composition may be a bivalent or a multivalent immunogenic composition, wherein the at least one nucleic acid sequence encoding the at least one Norovirus VP1 sequence and/or the at least one nucleic acid sequence encoding at least one Norovirus VP2 sequence may be selected from at least two different Norovirus genogroups, preferably a GII and a GI genogroups, and/or wherein the at least one nucleic acid sequence encoding the at least one Norovirus VP1 sequence and/or the at least one nucleic acid sequence encoding at least one Norovirus VP2 sequence may be selected from different Norovirus GII genotypes, preferably a GII.4 and a GII.17 or GII.2 genotype.

The combination of different GII genotypes, or the inclusion of a GI genogroup antigen together with a GII genogroup antigen can further enhance the therapeutic spectrum and may be of increasing importance facing the global spread of different Noroviruses to regions other than the region the virus was initially identified.

In a second aspect, there is provided a nucleic acid molecule, wherein the nucleic acid molecule may comprise at least a first and at least a second nucleic acid sequence, wherein the first nucleic acid sequence may encode at least one VP1 protein of a Norovirus or a functional fragment thereof, and wherein the second nucleic acid sequence may encode at least one VP2 protein of a Norovirus or a functional fragment thereof, optionally wherein the at least one first and the at least one second nucleic acid sequence is/are separated by at least one linker.

Based on the construction principle detailed above for the first aspect of the present invention, Norovirus constructs suitable to form a Norovirus VLP and thus suitable as antigen presenting entities for vaccination purposes may be designed rapidly in view of the rapid adaption and change of the Norovirus.

The co-expression of at least one VP1 and at least one VP2 encoding sequence can enhance the stability of the VLPs formed from the capsid proteins after recombinant expression or after immunization so that a reliable immune response can be triggered by the antigens such presented.

In one embodiment, the at least one first and/or the at least one second nucleic acid sequence may be independently selected from a Norovirus having a GII.4 genotype, preferably from a sequence from a Norovirus Hu/GII.4/Sydney/NSW0514/2012/AU, a Norovirus Hu/GII.4/Orange/NSW001 P/2008/AU, a Norovirus Hu/GII.4/NIHIC17.5/2012/USA, a Norovirus Hu/GII.4/G114-NG1242/2011/JP, or a Norovirus Hu/GII.4/Hiroshima4/2012/JP, wherein the at least one nucleic acid sequence encoding the at least one Norovirus VP1 and the at least one nucleic acid sequence encoding the at least one Norovirus VP2 may be derived from the same Norovirus, and/or wherein the at least one nucleic acid sequence encoding the at least one Norovirus VP1 and the at least one nucleic acid sequence encoding the at least one Norovirus VP2 may be derived from a different Norovirus, preferably selected from a Norovirus of a different genogroups, including genogroups GI, GII and GIV, and/or wherein the at least one nucleic acid sequence encoding the at least one Norovirus VP1 and/or the at least one nucleic acid sequence encoding at least one Norovirus VP2 are selected from different Norovirus GII genotypes, preferably a GII.4 and a GII.17 or GII.2 genotype. As already detailed above for the first aspect, relying on the basic cloning and construction principles disclosed herein allowing the provision of an immunogenic composition comprising both infectious virus particles and Norovirus VLPs as self-assembled in a functional form, other Norovirus VP1 and/or VP2 sequences can be defined.

In one specific embodiment, the nucleic acid molecule may comprise a sequence selected from any one of SEQ ID NOs:3 to 8, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or wherein the first nucleic acid sequence and/or the second nucleic acid sequence represent(s) a conserved consensus sequence.

There is no specific order in which the at least one VP1 and/or the at least one VP2 sequence have to be provided in a vector backbone as long as a functional full-length sequence can be expressed, which can then alone (VP1) or together with another partner (VP1 and VP2) assemble into a VLP presenting the relevant antigens.

In a third aspect according to the present invention, there is provided a recombinant virus-like particle (VLP), wherein the VLP may comprise at least one recombinant Norovirus VP1 antigen and at least one recombinant Norovirus VP2 antigen as defined according to the first aspect detailed above.

One advantage of the immunogenic compositions according to the present invention is the fact that the vector backbone itself provide immune-stimulation so that no additional adjuvant has to be provided in the immunogenic composition. For certain applications, however, it may be favorable to administer a composition which is not replicative, i.e. a VLP originating from a Norovirus. The present invention provides methods to produce both infectious virus particles as well as VLPs as well as methods to separate the two populations from each other to provide an immunogenic composition which comprises both populations, or to provide an immunogenic composition which comprises either infectious virus particles or VLPs. More than one VLP species may be present in certain embodiments, e.g. VLPs from a VP1 and VP2 protein, VLPs only comprising VP1 proteins, or compositions comprising several VLPs of different constitution, e.g. when using several different VP1 and/or VP2 sequences, for example, in a monovalent vaccine. Immunogenic compositions exclusively comprising VLPs can be further provided together with at least one pharmaceutically acceptable adjuvant.

In certain embodiments, the ratio between infectious virus particles (IVPs) and the at least one VLP, which may have the same or a different composition of VP1 and/or VP2 Norovirus antigens, may be specifically balanced. I.e., the ratio of IVPs to VLPs may be 1:1; 1:5; 1:10; 1:20; 1:50; 1:100; 1:500; 1:1,000; 1:5,000; 1:10,000 and the like. A surplus of VLPs in the composition may be suitable in certain patient/subject groups. Still, the presence of IVPs, at least to a certain extent, can be favorable in view of the self-adjuvanting effect thereof. Based on the methods disclosed herein, immunogenic compositions comprising a specific ratio of IVPs and VLPs can be produced in a reliable manner. In certain embodiments, where a strong immune response, in particular a string initial innate immune response is required, the amount of IVPs may be greater (in terms of ration) than the number of VLPs. In certain embodiments, only IVPs or only VLPs may be provided.

In another aspect, there is provided a host cell, wherein the host cell may comprise at least one nucleic acid sequence encoding at least one Norovirus VP1 sequence and at least one Norovirus VP2 sequence according to the first aspect above, or wherein the host cell may comprise at least one nucleic acid molecule according to the second aspect disclosed above, or wherein the host cell may comprise at least one nucleic acid sequence encoding a recombinant virus-like particle (VLP) according to the third aspect, preferably wherein the at least one host cell is selected from the group consisting of Vero cells, chicken embryo fibroblast cells, HEK293 cells, HeLa cells, fetal rhesus lung cells or MRC5 cells. Other suitable host cells are available to the skilled person. The host cell has to be compatible with a propagation of the vector backbone chosen.

In yet a further aspect according to the present invention, there is provided a method for producing an immunogenic composition as disclosed herein, or for producing a recombinant virus-like particle (VLP) as disclosed herein, wherein the method may comprise the following steps: (i) inserting at least one nucleic acid sequence as defined in the context of the immunogenic compositions disclosed herein, or inserting at least one nucleic acid molecule as disclosed herein, into a vector backbone as defined in the context of the first aspect disclosed herein to operably link the nucleic acid sequence or molecule and the vector backbone to obtain a recombinant chimeric virus sequence, wherein chimeric implies that the vector backbone and the insert originate from different viral sequences and may additionally be modified or codon-optimized; (ii) infecting at least one host cell with at least one recombinant chimeric virus sequence obtained in step (i) to obtain a virus sample, wherein the virus sample may comprise a mixture of infectious virus particles and VLPs; (iii) optionally: clarifying the virus sample of step (ii); (iv) optionally: purifying the clarified virus sample of step (iii); (v) optionally: formulating the at least one recombinant chimeric virus with at least one pharmaceutically and/or veterinary acceptable carrier and/or excipient; (vi) obtaining an immunogenic composition comprising infectious virus particles and virus-like particles (VLPs); (vii) optionally: further purifying the immunogenic composition and thus obtaining, separated from each other, recombinant virus-like particles (VLPs) and/or infectious virus particles.

The term “clarifying” or “clarification” according to the present disclosure refers to a step for removing large impurities from a bulk product such as a cell culture supernatant to be clarified. The term removing also encompasses a reduction in the amount (e.g. concentration) of impurities. Clarification may rely on separation by size, molecular weight or density and the like. “Large impurities” is to be taken to preferably refer to a size bigger than, for example, sterically demanding MV particles. Clarification thus aims at removing cells and cell debris from the host cells infected with and producing a virus and/or VLPs. Clarification usually does not include separation of impurities that have a size corresponding to the virus particles to be purified or smaller. Common methods for clarification are centrifugation and microfiltration, including tangential flow filtration (TFF), ultracentrifugation, dead end filtering and the like. In the context of clarifying a bulk product comprising MV particles centrifugation is less desired, as centrifugation processes are not easily scalable under GMP conditions.

Preferably, chromatography, in particular flow-through chromatography may be used for purification. Ac chromatography unit at least comprises a stationary phase material that is preferably selected from the group consisting of at least a resin, at least a matrix, at least a gel and, preferably, beads. In a preferred embodiment, the stationary phase material is comprised in a container, e.g. for carrying out column chromatography. In preferred embodiments, the stationary phase material, preferably the beads, has/have at least size-exclusion functionality, hydrophobic interaction functionality or ion exchange functionality, or a combination thereof, preferably at least size-exclusion functionality, optionally combined with hydrophobic interaction functionality. The stationary phase material, preferably the beads, may comprise a ligand-activated core, and an inactive shell comprising pores. In another embodiment, the stationary phase may have a monolithic arrangement.

In certain embodiments according to the above method of the present invention, there is further provided a method additionally comprising a further purification step, comprising: further purifying the recombinant chimeric virus and/or VLPs as pharmaceutically active constituents by means of filtration, centrifugation, tangential flow filtration, membrane filtration, purification with grafted media, aqueous two phase extraction, precipitation, buffer exchange, dialysis or chromatography, including size exclusion chromatography for separating the purified recombinant infectious virus particles derived from a measles virus into a fraction containing virions and another fraction containing virus-like particles or recombinant subviral particles. Said embodiment is especially useful in case the antigenic region inserted as nucleic acid sequence into an attenuated vector backbone, preferably a measles virus vector backbone, has to be further separated into a fraction containing the replication competent virions of the measles virus containing genetic material, and the VLPs self-assembled from the antigens of the at least one Norovirus, preferably from at least one VP1 and at least one VP2 protein from a Norovirus, wherein the VLPs are devoid of any genetic material. Therefore, they possess relevant surface antigens to elicit an immune response, preferably a protective immune response, but cannot further be propagated in a host cell, which makes VLPs and other subviral particles an interesting target for several applications in immunology.

In view of the rather pronounce difference in the size and physico-chemical properties of infectious measles virus particles and VLPs derived from at least one Norovirus, specific purification strategies have to be applied as further detailed in Examples 4 and 5 below. In yet a further embodiment, there is provided an immunogenic composition according to the first aspect, or an immunogenic composition comprising at least two VP1 antigens from different Noroviruses, or a recombinant virus-like particle (VLP) to the third aspect disclosed herein for use in a method of preventing or treating a Norovirus virus disease in a subject.

As detailed above and as further detailed in the below Examples, the immunogenic compositions and the VLPs disclosed herein are suitable in triggering a protective immune response in a subject. The VLPs generated and comprised by the immunogenic compositions are stable and highly efficient in presenting the Norovirus antigens to elicit a protective immune response whilst having low reactogenicity.

In a further aspect, an immunogenic composition according to the first aspect, or an immunogenic composition comprising at least two VP1 antigens from different Noroviruses, or a nucleic acid molecule according to the second aspect, or a recombinant virus-like particle (VLP) according to the third aspect for use in a method of preventing or treating a Norovirus virus disease in a subject. A method for preventing or treating a Norovirus disease in a subject may thus be provided which allows the prevention or reduction of the symptoms of a Norovirus infection in a subject, i.e. a prevention or reduction of the symptoms of acute gastroenteritis in all age groups to reduce severe disease burden and thus complications in infected populations, in particular amongst children and elderly people, for which dehydration associated with Norovirus infection is a huge problem and associated with high rates of hospitalization and severe complications.

The present invention is further described with reference to the following non-limiting examples.

### EXAMPLES

### Example 1: Norovirus Antigen Design

For vaccine design, it was a main intention to design a versatile cloning strategy allowing the rapid provision of Norovirus vaccines in a modular manner. In view of the fact, on a global scale, different Norovirus genotypes and genogroups are predominating in different climate zones and populations, a vaccine should be created covers the most severe recently pandemic Norovirus genotypes and that can be adapted at short notice to keep up with Norovirus evolution.

Noroviruses are non-enveloped and belong to the Family of Caliciviridae. The genus is divided into 6 genogroups of which genogroup I, II and IV contain primarily human Noroviruses. The genome is organized in three open reading frames (ORF). Noroviruses produce two capsid proteins, the major capsid protein VP1 encoded by ORF2 and the minor capsid protein VP2 encoded by ORF3. These two proteins are translated from a subgenomic RNA. The outer capsid shell consists of the major capsid protein VP1. This protein has the ability to self-assemble into virus-like particles (VLPs), which morphologically and antigenically resemble the native virus. Therefore, VLPs are highly immunogenic and are the best candidate for vaccine development. No additional protein is needed for the VLP formation. Nevertheless, the minor capsid protein VP2 if expressed together with VP1 can also be incorporated into the virus-like particles. VP2 stays within the capsid, on the inner side of the capsid shell, and is thus not target of the immune response.

To take all this information into account, a Norovirus vaccine candidate based on the expression of either VP1 alone and VP1 and VP2 was developed. In a first instance, the coding sequence of VP1 and VP2 were chosen. As these proteins are not expressed as one polyprotein that is co- or post-translationally cleaved by host proteases, a transcriptional terminator-promotor region was introduced between the two coding sequences to ensure that both proteins are expressed and are thus available for VLP formation. The measles-specific terminator-promotor region located between P and M was taken and inserted in between (cf. SEQ ID NOs: 9 for VP1 and SEQ ID NO:10 for VP1 and VP2 both cloned into a specifically adapted ATU of the measles virus antigenomic sequence). Furthermore, it was crucial to identify a suitable backbone sequence in advance. The measles backbone as disclosed herein has several advantages. First, the vector backbone per se is safe and established. When properly taking into consideration the specific needs of this vector, it can be used as efficient shuttle platform to produce infectious virus particles based in the measles backbone as well as VLPs mainly as determined by the specific Norovirus insert to allow a rapid vaccine production.

For cloning, a Kozak sequence was also added in front of Norovirus insert sequence (cf. SEQ ID NOs:9 and 10) in order to obtain an optimal protein translation in target cells. This step, however, may be optional in view of the fact that Norovirus sequence usually express well in mammalian cell systems. In order to avoid read-through during protein translation, more than one stop codon was added at the end of the Norovirus antigens. Two or three stop codons were added to obey the rule of six as demanded by the measles backbone chosen as vector, respectively. This rule of six is especially important, if measles virus-derived RNA is used as a vector backbone, as otherwise no replication would be initiated. If working with other vector backbones, other design parameters have to be considered.

As a proof of principle of the general applicability of the cloning technique designed, two further constructs were established. First of all, it was the idea to create a Norovirus vaccine that also provides protection against severe non-GII.4 genotypes, for example, as those genotypes implicated in epidemics in China and Hong Kong, where a bimodal seasonality of Norovirus is observed, nowadays often also caused by non-GII.4 genotypes like GII.17 affecting all age groups GII.2 affecting older children an younger adults. Therefore, VP1 and VP2 genes of non-GII.4 genotypes, for example, as disclosed in Chi-Wai et al. (Bimodal Seasonality and Alternating Predominance of Norovirus GII.4 and Non-GII.4, Hong Kong, China, 2014-2017, CDC EID Journal, vol. 24, 2018) were cloned and codon-optimized. Notably, monovalent (GII.2 only and GII.17 only) and bivalent (GII.4 VP1 or VP2 and correspondingly a different VP1 or VP2 gene from a GII.2 or GII.17 genotype) were cloned. Another interesting candidate is a VP1 or VP2 from a GI genotype, i.e. that genotype the “prototypic” Noro-Norwalk virus belongs to. Fortunately, new Norovirus sequences are sequenced and published rapidly so that new potentially antigenic sequences can be retrieved ad hoc from various accessible databases so that the sequences can be further designed in silico and can be sent to synthesis afterwards without the need for a biological material of a Norovirus infected patent as template.

Another idea was to define a generally applicable consensus sequence derived from pandemic Norovirus. This strategy relies on the comparison of VP1 and VP2 sequences from at least two or three genotypes which have been identified as relevant cause of Norovirus outbreaks. An example of this strategy is presented in FIGS. 1 and 2. First, the sequences are retrieved from the relevant databases and are then subjected to an alignment, preferably a sequence alignment optimized for high accuracy, e.g. MUSCLE. The date thus obtained can be immediately analyzed by MView (version 1.63). As evident from FIG. 1B and FIG. 2B, this allows a graphical overview of highly conserved amino acid sequences within the different genotype sequences analyzed. Highly conserved sequences, i.e. sequences conserved in all compared sequences, should thus always be present in a consensus sequence. Other, non or less conserved sequences can be designed to obtain optimum protection against different genotypes by combining prominent amino acid residues of different characteristic, for example, in FIG. 1B, position 80 being a rather positively charged arginine in SEQ ID NO:12, and a likewise positively charged lysine in SEQ ID NOs:2, 14 and 16. Therefore, a positively charged residue (+) should be introduced into the consensus sequence. Interestingly, position 92 is a phenylalanine (F) in SEQ ID NO:2, but a serine in the other sequences compared in FIG. 1. S and F, however, have completely different properties. Therefore, a consensus sequence could be used relying on either the S or the F residue to test which sequence provides optimum protection. For example, a S could be integrated at the relevant position in an otherwise SEQ ID NO:2 based sequence to create chimeric sequences providing optimum protection against different Norovirus genotypes.

### Example 2: Codon Optimization, Mutagenesis and Vector Construction

The selected Norovirus antigens were subjected to codon optimization. Again, this might be an optional step in view of the fact that Norovirus is perfectly adapted to be expressed in a mammalian/human host cell. Codon optimization to adapt the codon usage of the desired nucleic acid sequences to a human codon usage was performed by Eurofins Genomic. MV-editing sequences were avoided. Cloning of synthesized Norovirus inserts (synthesis: Eurofins Genomics) into a recombinant vector backbone was performed using standard techniques of molecular biology. Preferably, inserts were cloned into the vector backbone derived from a measles virus Schwarz strain into the ATU2 region using the restriction enzymes BsiWI and BssHII.

### Example 3: Infection and Master Cell Bank (MCB) and Rescue

For the purpose of this example, a recombinant, Norovirus antigen expressing, live attenuated chimeric virus (see SEQ ID NOs:9 and 10) was used which was generated by transfecting helper cells with the vector, wherein said helper cells (the human embryonic kidney cell line 293 was chosen because it is highly permissive for MV. In addition, these cells can be transfected efficiently, e.g. by the calcium phosphate co-precipitation method) are capable of expressing helper functions to express an RNA polymerase, and to express the N, P and L proteins of a MV virus); co-cultivating said transfected helper cells of step 1) with passaged cells suitable for the passage of the MV attenuated strain from which the cDNA originates; 3) recovering the recombinant infectious virus expressing at least one structural protein of a Norovirus to provide a master virus seed stock (MVSS). Vero 10-87 cells served as host cells and as master cell bank (MCB). The MVSS used for this exemplary purification scheme can be obtained from the sequence according to SEQ ID Nos:9 and 10 according to the present application.

Details on the rescue procedure for measles virus based MVSSs can be taken from Radecke et al. (Rescue of measles viruses from cloned DNA EMBO J. 1995 Dec. 1; 14(23): 5773-5784).

### Example 4: MCB Revival, Expansion, Harvest and Purification Under GMP Conditions

For vaccine production, sterile and reproducible production strategies are needed, in particular when working with potentially Two cryovials each containing 1.0 mL MCB were removed from storage in vapour phase liquid nitrogen and are transported to the cleanroom in a sanitised container which is in turn transported on dry ice. Once within the cleanroom, the MCB cryovials were thawed in hands/at ambient while gently swirling the content until all ice within the vials has melted. When the vials had thawed they were transferred to a biosafety cabinet. The thawed cell suspensions from each vial were transferred aseptically into 50 mL centrifuge tubes. 9 mL of pre-warmed Dulbecco's Modified Eagle Medium (DMEM)+10% fetal bovine serum (FBS) medium were added drop-wise to each 50 mL tube containing the thawed cells while gently swirling the tubes. The MCB/WCB cryovials were each rinsed with the homogeneous cell suspension from the centrifuge tube and the rinse was transferred back to the respective 50 mL tubes.

The cell suspensions were then centrifuged at 300×g±5% for 5 minutes at room temperature. The supernatants were discarded and the pellets were suspended in 10 mL DMEM+10% FBS medium. The resuspended pellets were removed from the biosafety cabinet and centrifuged for a second time using the same parameters as before. The supernatant was again discarded and the pellets were resuspended in 10 mL DMEM+10% FBS medium. 0.5 mL from the prepared cell suspensions was removed and used to perform a cell count determining viable cells and viability. The remaining cell suspensions were passaged to one T225 cell culture flasks/suspension and medium was made up to 50 mL using pre-warmed DMEM+10% FBS medium. Following growth in flask culture, the supernatant from the 2×T225 flasks was removed, discarded and the cell monolayer was washed with pre-warmed D-PBS. Pre-warmed TrypLE Select was added to each flask and distributed evenly over the monolayer. Flasks were incubated to detach cells and then observed for cell detachment under the microscope. If necessary, the flasks were taped gently for cell detachment. If detachment was below 90%, flasks were further incubated until detachment of greater than 90% was reached.

Pre-warmed DMEM+10% FBS medium was added to each flask and the cell suspension was removed to sterile centrifuge tubes. After centrifugation, the supernatant was removed and discarded while the cell pellets were resuspended in DMEM+10% FBS medium by pipetting up and down. The cultures were fully suspended if no cell clumps were visible. Resuspended cell-solutions from 2×T225 flasks were each transferred into sterile containers (50 mL centrifugation tube) and mixed to obtain a homogenous solution of cells. 0.5 mL from the prepared cell suspensions were removed and used to perform a cell count determining viable cells and viability of each cell solution. The remaining cell suspensions were passaged to 5× T225 cell culture flasks/suspension and medium was made up to 50 mL using pre-warmed DMEM+10% FBS medium. Thus, in total 10×T225 flasks were prepared in this exemplary setting, which might naturally depend of the nature of the product to be produced.

Following growth in flask culture stage 2, the supernatant from the two T225 sets flasks was removed, discarded and the cell monolayer was washed with pre-warmed D-PBS. Pre-warmed TrypLE Select was then added to each flask and distributed evenly over the monolayer. Flasks were incubated to detach cells and then observed for cell detachment under the microscope. If necessary, the flasks were taped gently for cell detachment. If detachment was below 90%, flasks were further incubated until detachment of greater than 90% was reached.

Pre-warmed DMEM+10% FBS medium was added to each flask and the cell suspensions were removed to sterile centrifuge tubes. The cell suspensions were then centrifuged. After centrifugation, the supernatant was removed and discarded while the cell pellets were resuspended in DMEM+10% FBS medium by pipetting up and down. The cultures were fully suspended if no cell clumps were visible. Resuspended cell-solutions from each set (5×T225 flasks) were transferred/pooled into one sterile container (50 mL centrifugation tube) and mixed to obtain a homogenous solution of cells. 0.5 mL from the prepared cell suspensions were removed and used to perform a cell count determining viable cells and viability of each cell solution. After analysis of viable cells and viability, the best performing set of T225 flasks was selected and passaged to 30× T225 cell culture flasks and medium was made up to 50 mL using pre-warmed DMEM+10% FBS medium.

Details on the further cultivation, expansion, cell culture infection and processing until harvest and a subsequent purification was performed according to the protocols as detailed in the International patent applications WO 2017/109211 A9 and WO 2017/109222 A1 as well as disclosed in European patent application EP18178037.0 the content of which is incorporated herein by reference. A particular problem for chromatographic purification approaches may be pleomorphic viruses, i.e., the appearance and shape of one and the same virus can vary within a given preparation. MV is such as pleomorphic virus. Even though the Norovirus VLPs forming by self-assembly will be much smaller, purification of infectious chimeric MV particles, said infectious MV particles as vector backbone also encoding the genetic information for foreign Norovirus antigens to be expressed as VLPs according to the present disclosure, should thus follow special purification strategies as detailed herein below in Example 5.

Typically, following growth in flask culture, the supernatant from the 30×T225 flasks will be removed, discarded and the cell monolayer will be washed with pre-warmed D-PBS. Pre-warmed TrypLE Select can be added to each flask and distributed evenly over the monolayer. Flasks can be incubated to detach cells and observed for cell detachment under the microscope. If necessary, the flasks can be taped gently for cell detachment. If detachment is below 90%, flasks were further incubated until detachment of greater than 90% was reached.

Pre-warmed DMEM+10% FBS medium was added to each flask and the cell suspension can be removed to sterile centrifuge tubes. After centrifugation, the supernatant can be removed and discarded while the cell pellet is resuspended in DMEM+10% FBS medium by pipetting up and down. Resuspended cell-solutions from T225 flasks can be transferred into one sterile container and will be mixed to obtain a homogenous solution of cells. 0.5 mL from the prepared cell suspension should be removed and should be used to perform a cell count determining viable cells and viability. Spinner Flasks and Bioreactor microcarrier assisted culture can follow ONO 2017/109222 A1).

Cells should be ≥80% viable and microcarrier ≥80% confluent. The cell count can be used to determine the number of viral particles required to infect the culture at an MOI of 0.01 TCID₅₀/cell. This MOI may again vary depending on the host cell and the MVSS chosen, but can be easily determined after standard pre-testings with the respective host cell and the respective virus. MOIs between 0.0001 and 0.1 are preferable. Notably, the time point for harvest will change depending on the chosen MOI, which can be determined by the skilled person. After calculating the required amount of virus, an appropriate number of viral vials can be removed from −80° C. storage and they can be transported to the cleanroom. The virus vials are then thawed at ambient/in hands until all ice has melted. The virus volume required to infect a 10 L suspension at an MOI of 0.01 TCID₅₀/cell is calculated and is diluted in 5000 mL VP-SFM w/o phenol red.

### Example 5: Further Separation of VLPs from Infectious MV Particles

The resulting material of the above expression, harvest and downstream processing is an immunogenic composition comprising both, infectious MV particles as well as self-assembled VLPs of Norovirus even though it was observed that a huge proportion of the material already represented Norovirus VLPs expressing both VP1 and VP2.

For certain applications, it may be just the infectious and replicative virus, which is intended for use as vaccine. For other applications, e.g. in children, it may be preferred to further separate MV particles from VLPs. This can be achieved by performing a further round of column purification using a suitable column material. In case the VLPs are the product of interest, the huge and pleomorphic MV backbone may simply be adsorbed to a column material and the VLPs may be obtained as flow-through. In view of the specific physico-chemical properties of the MV and the Norovirus VLPs representing non-enveloped VLPs a suitable column material for a high yield recovery of VLPs, if desired, has to be determined. In particular an initial column chromatography purification as disclosed in WO 2017/109211 A9 relying on monolithic columns can potentially separate MVs from Norovirus VLPs during the initial chromatographic step. A process as disclosed EP18178037.0, depending on the specific process parameters, will likely not allow the VLP Norovirus particles to interact with the ligands using a CaptoCore material so that VLPs and MV will be obtained in the flow-through. Still, this may depend on the nature of the VLPs to be purified. Depending on the strongly varying size and surface characteristics of MV and Norovirus VLPs, an additional purification step can then be added to separate MVs from Norovirus VLPs to obtain both fractions in a highly purified form, if desired.

The diluted virus solution comprising a mixture of MV and Norovirus VLPs may thus be—in a GMP mode—connected to one of the bypass lines of inlet S1, using the SCD. The bypass on S1 is used to remove air from the line. It has to be ensured that no air will be introduced into inlet S1 later on. Otherwise only part of the virus material will be processed on the respective column. Depending on the resolution and the parameters chosen for column purification and depending on the column material, e.g. a size exclusion column, either one product peak will be obtained comprising the recombinant chimeric MV virus particles derived from a measles virus backbone as scaffold including nucleic acid material from a Norovirus packaged therein and optionally VP1/VP2 virus-like particles and VP1 VLP particles. Alternatively, two or three separate peaks can be obtained, one comprising the recombinant infectious virus particles and the other one comprising VLPs of VP1 and VP2 and one peak comprising VP1 VLPs (which may also form spontaneously). In the case of co-elution, a mixed population can optionally be further purified, polished or subjected to a buffer-exchange (for further details, see WO 2017/109211 A9, WO 2017/109222 A1 and EP18178037.0). At the end of the purification cycle, the exact volume of main peak fraction is noted and the bulk virus pool is snap frozen at −80° C.±10° C. directly or aliquot as required prior freezing. Alternatively, part of the virus pool can immediately be subjected to further analysis, including analysis for purity and infectivity and the like. The main peak fraction can additionally be subjected to a further round of purification, polishing or buffer-exchange to separate recombinant infectious virus particles containing genetic material from virus-like particles. Using the SCD, 20% EtOH was connected to buffer inlet B3. The system was flushed with 20% EtOH. Using the SCD, all buffers from the Äkta Pilot inlets were disconnected and discarded, the T-piece on inlet S1 was disconnected and discarded and a cleaning tubing was connected to inlets S1, S2, A1, A2, B1, B2 and B3. The cleaning inlet tubing was connected to 1 M NaOH. All inlets, outlets and the system were cleaned with 1 M NaOH. The 1 M NaOH connected to the cleaning tubing was replaced by WFI. Flush the system with WFI. Replace the WFI connected to the cleaning tubing by 20% EtOH. Store the Äkta Pilot in 20% EtOH.

### Example 6: Characterization and Immunogenicity Testing

MV Noro viruses were rescued and their biological properties were determined in cell culture. First, growth properties of MV Noro were analyzed and compared to the parent MV Schwarz. Growth curve analyses were performed on Vero 10-87 cells. Therefore, the cells were infected with a defined MOI (MOI of 1, 0.1 or 0.01) and samples were taken at different time points. MV-Noro exhibited slower growth kinetics than the MV Schwarz parent strain as determined by TCID50 and/or qPCR. Adaptation of different growth parameters increases the virus yield and allows the production of infectious virus particles similar to the MV Schwarz parent strain.

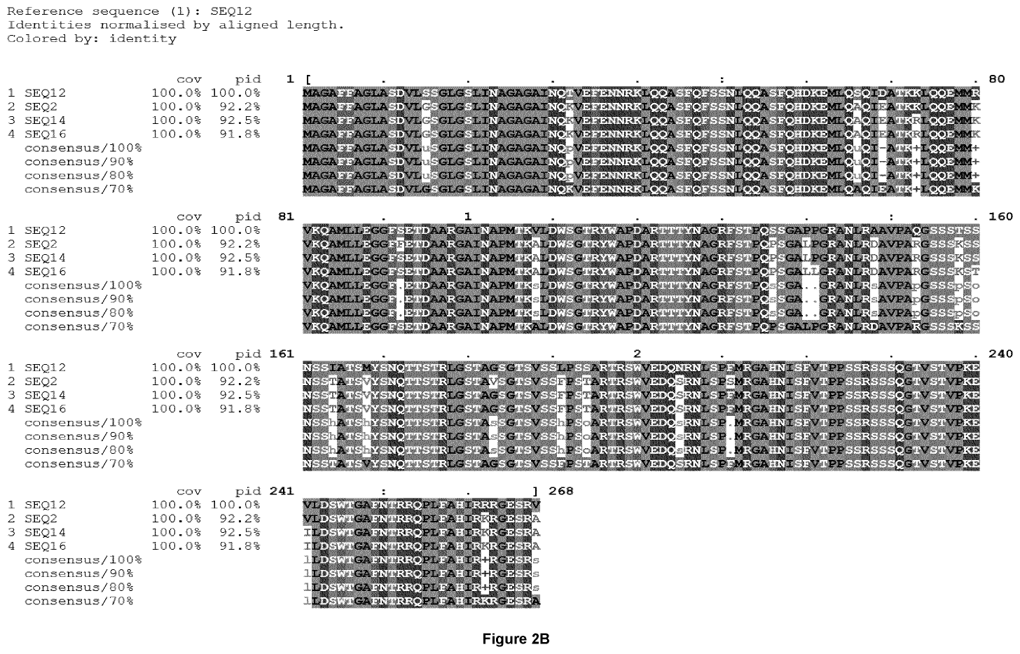

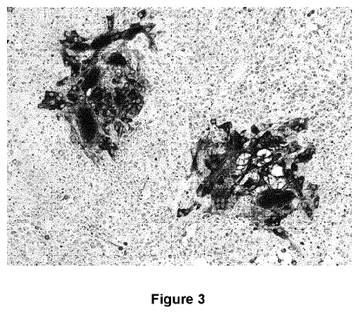

The antigen expression of the Noro VP1 protein was determined by immunostaining. Vero 10-87 cells were thus infected with a defined MOI (1, 0.1 or 0.01). Cells were stained with a commercially available Norovirus GII.4 antibody recognizing Norovirus virus-like particle (VLP) from GII.4 VLP 1987-2009 and 2012. Depending on the MOI used, cells exhibited a strong staining for Norovirus VP1 protein indicating that virus-like particles can be successfully expressed in functional form from the measles virus vector backbone. Expression of Norovirus VLPs was visualized and verified as shown in FIG. 3.

### Example 7: Animal Model

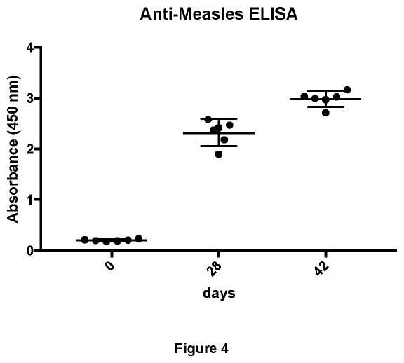

To evaluate the immunogenicity of the MV Noro vaccine candidate in an animal model, groups of cotton rats were vaccinated intramuscularly with various doses of MV Noro (VP1+VP2, SEQ ID NO:10). All cotton rats were given a homologous booster immunization four weeks (28 days) after the first immunization. Sera were collected at various time points and analyzed for the presence of anti-Norovirus and anti-measles virus specific antibodies. Measles antibodies were detected by using a commercially available EUROIMMUN kit with slight modifications. A secondary antibody labelled with peroxidase specific for cotton rats was used instead of the human one. All cotton rats seroconverted to measles after the first immunization as shown in FIG. 4 indicating that cotton rats are a valid animal model to test recombinant measles-vectored vaccine candidates.

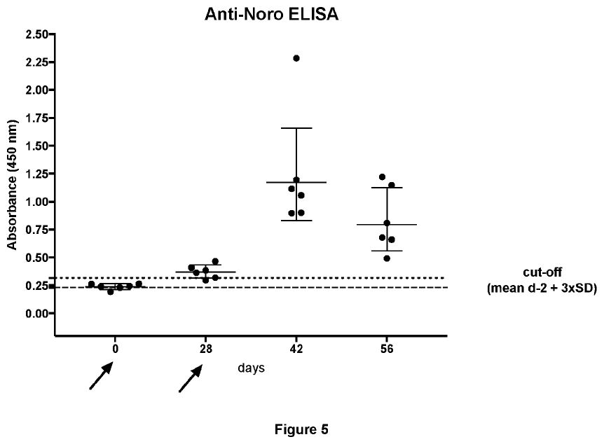

For the detection of Norovirus specific antibodies, an in-house ELISA was developed as follows. Plates were coated with recombinant GII.4 VLP overnight. On the next day, sera were pre-diluted and added to the coated plates. After a certain incubation period, the plates were washed and a secondary incubation step with the same secondary cotton rat antibody as used for the measles ELISA was initiated. Finally, substrate was added and after a certain time the reaction was stopped and the extinction values were measured and blotted on a graph (FIG. 5). As evident from FIG. 5 showing four different measurement points (day zero before prime immunization, 28 days post infection before boost immunization, day 42 and day 56), a slight increase in Norovirus specific antibodies was already observed after the prime vaccination. The boost vaccination then resulted in a pronounced increase in antibody responses in sera from all tested animals.

### Example 8: Carbohydrate-Binding Blocking Assay

The blocking assay is used for identification of Norovirus specific antibodies that functionally block binding of Norovirus capsid proteins to carbohydrate ligands, HBGA. Sera that were tested positive for the presence of Norovirus-specific antibodies in the ELISA were further subjected to this analysis. These antibodies have been confirmed by the in-house ELISA to be able to recognize and bind GII.4 VLPs.

An antibody blocking assay was performed using synthetic carbohydrate HBGAs. Microtiter plates were coated overnight at 4° C. with synthetic HBGAs. On the next day, GII.4 VLP and serially diluted sera were pre-incubated for a certain time and added to the coated plates. As a next step, VLPs bound to the HBGA ligand were detected by using first a polyclonal GII.4 VLP serum followed by a secondary HRP labelled antibody. The extinction values 450 nm were measured by an ELISA reader. An OD reading from the wells lacking cotton rat serum of the immunogenicity study was considered as a maximum signal for the binding of VLPs to the synthetic HBGAs further meaning that no antibody blocking could be observed. Blocking index was calculated as the percentage of the maximum blinding blocked by a certain serum dilution. As a result, the vaccine candidate was able to induce the production of functional Norovirus specific antibodies that block binding of VLPs to HBGA.

### Example 9: Sucrose Gradient and Western Blotting

To further optimize Norovirus VLP purification, VLPs as obtainable from SEQ ID NO:10, i.e. VLPs comprising both VP1 and VP2 sequences, were produced as follows. Vero cells were infected with MV-Noro virus and 6 days post infection cell culture supernatants were harvested and clarified by 3,000×g for 30 min at 4° C. Then, recombinant measles virus and Norovirus VLPs in the supernatant were concentrated by ultracentrifugation and pellets were resuspended in Lysis Buffer, thereby destroying the cell membranes of the virus particles. The lysis buffer containing VLPs was then subjected to a sucrose cushion and put to high speed centrifugation. The difference in densities between sucrose and lysis buffer allowed the separation of different cell components. The sucrose layer contained the VLPs. A fraction was further analyzed by SDS Page and Western Blot.

1. An immunogenic composition comprising at least one recombinant Norovirus antigen, and/or a sequence encoding the same, wherein the at least one recombinant Norovirus antigen is encoded by at least one nucleic acid sequence encoding at least one Norovirus VP1 sequence and at least one Norovirus VP2 sequence, wherein the at least one nucleic acid sequence is operably linked to a vector backbone, and wherein the at least one nucleic acid sequence encoding the Norovirus VP1 sequence and the at least one nucleic acid sequence encoding the at least one Norovirus VP2 sequence is separated by at least one artificial linker sequence having regulatory or spacer functionality, wherein the immunogenic composition optionally comprises at least one pharmaceutically acceptable carrier and/or excipient.

2. The immunogenic composition according to claim 1, wherein the Norovirus VP1 sequence and/or the Norovirus VP2 sequence are independently derived from a Norovirus of the GII genogroup.

3. The immunogenic composition according to claim 1, wherein the vector backbone is selected from the group consisting of a measles virus backbone, a lentivirus backbone, a Vesicular stomatitis virus (VSV) backbone, and a vaccinia Ankara backbone.

4. The immunogenic composition according to claim 1, wherein (a) the VP1 and/or the VP2 sequence are independently selected from a Norovirus having a GII.4 genotype; (b) the VP1 and the VP2 sequence are derived from the same Norovirus; (c) the VP1 and the VP2 sequence are derived from a different Norovirus; or (d) the VP1 and the VP2 sequence are modified in comparison to the reference sequence.

5. The immunogenic composition according to claim 1, wherein the at least one VP1 and/or the at least one VP2 sequence represent(s) a conserved consensus sequence.

6. The immunogenic composition according to claim 1, wherein the vector backbone comprises a nucleotide sequence of the full-length, infectious antigenomic (+) RNA strand of a measles virus, optionally wherein the immunogenic composition comprises a sequence of SEQ ID NO:10, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto.

7. The immunogenic composition according to claim 1, wherein the at least one recombinant Norovirus VP1 antigen is selected from an amino acid sequence of SEQ ID NOs:1, 11, 13 or 15 or a nucleic acid sequence encoding the same, or an amino acid sequence having at least 90%, 91%, 92%, 93%, 94%, 95%, 96%, 97%, 98% or 99% sequence identity thereto, and wherein the at least one recombinant Norovirus VP2 antigen is selected from an amino acid sequence of SEQ ID NOs:2, 12, 14 or 16 or a nucleic acid sequence encoding the same, or an amino acid sequence having at least 90%, 91%, 92%, 93%, 94%, 95%, 96%, 97%, 98% or 99% sequence identity thereto.

8. The immunogenic composition according to claim 1, wherein the immunogenic composition is a bivalent or a multivalent immunogenic composition, wherein the at least one nucleic acid sequence encoding the at least one Norovirus VP1 sequence and/or the at least one nucleic acid sequence encoding at least one Norovirus VP2 sequence are selected from at least two different Norovirus genogroups.

9. A nucleic acid molecule comprising at least a first and at least a second nucleic acid sequence, wherein the first nucleic acid sequence encodes at least one VP1 protein of a Norovirus or a functional fragment thereof, and wherein the second nucleic acid sequence encodes at least one VP2 protein of a Norovirus or a functional fragment thereof, wherein the at least one first and the at least one second nucleic acid sequence is/are separated by at least one artificial linker.

10. The nucleic acid molecule according to claim 9, wherein (a) the at least one first and/or the at least one second nucleic acid sequence is independently selected from a Norovirus having a GII.4 genotype; (b) the at least one first and/or the at least one second nucleic acid sequence is independently selected from a sequence from a Norovirus Hu/GII.4/Sydney/NSW0514/2012/AU, a Norovirus Hu/GII.4/Orange/NSW001P/2008/AU, a Norovirus Hu/GII.4/NIHIC17.5/2012/USA, a Norovirus Hu/GII.4/GII4-NG1242/2011/JP, or a Norovirus Hu/GII.4/Hiroshima4/2012/JP; (c1) the at least one nucleic acid sequence encoding the at least one Norovirus VP1 and the at least one nucleic acid sequence encoding the at least one Norovirus VP2 are derived from the same Norovirus, or (c2) wherein the at least one nucleic acid sequence encoding the at least one Norovirus VP1 and the at least one nucleic acid sequence encoding the at least one Norovirus VP2 are derived from a different Norovirus; (d) the at least one nucleic acid sequence encoding the at least one Norovirus VP1 and the at least one nucleic acid sequence encoding the at least one Norovirus VP2 are derived from a Norovirus of different genogroups, including genogroups GI, GII, and GIV; and/or (e) the at least one nucleic acid sequence encoding the at least one Norovirus VP1 and/or the at least one nucleic acid sequence encoding at least one Norovirus VP2 are selected from different Norovirus GII genotypes; and/or (f) the at least one nucleic acid sequence encoding the at least one Norovirus VP1 and/or the at least one nucleic acid sequence encoding at least one Norovirus VP2 are selected from a GII.4 and a GII.17 or GII.2 genotype.

11. The nucleic acid molecule according to claim 9, wherein the nucleic acid molecule comprises a sequence selected from any one of SEQ ID NOs:3 to 8, or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity thereto, or wherein the first nucleic acid sequence and/or the second nucleic acid sequence represent(s) a conserved consensus sequence.

12. A recombinant virus-like particle (VLP) comprising at least one recombinant Norovirus VP1 antigen and at least one recombinant Norovirus VP2 antigen, wherein the at least one recombinant Norovirus VP1 antigen and/or the at least one recombinant Norovirus VP2 antigen (a) are independently derived from a Norovirus of the GII genogroup; (b) are independently selected from a Norovirus having a GII.4 genotype; (c) are derived from the same Norovirus; (d) are derived from two different Norovirus genotypes; (e) are modified in comparison to the reference sequence; (f) represent(s) a conserved consensus sequence; or (g) are derived from a GII.4 Norovirus genotype and a GII.17 or a GII.2 Norovirus genotype.

13. A host cell comprising at least one nucleic acid sequence encoding at least one Norovirus VP1 sequence and at least one Norovirus VP2 sequence according to claim 1, optionally wherein the host cell is selected from the group consisting of a Vero cell, a chicken embryo fibroblast cell, a HEK293 cell, a HeLa cell, a fetal rhesus lung cell, and a MRC5 cell.

14. A method for producing an immunogenic composition according to claim 1, the method comprising the following steps:
(i) inserting at least one nucleic acid sequence as defined in claim 1 into a vector backbone as defined in claim 1 to operably link the nucleic acid sequence or molecule and the vector backbone to obtain a recombinant chimeric virus sequence;
(ii) infecting at least one host cell with at least one recombinant chimeric virus sequence obtained in step (i) to obtain a virus sample;
(iii) optionally: clarifying the virus sample of step (ii);
(iv) optionally: purifying the clarified virus sample of step (iii);
(v) optionally: formulating the at least one recombinant chimeric virus with at least one pharmaceutically and/or veterinary acceptable carrier and/or excipient;
(vi) obtaining an immunogenic composition comprising infectious virus particles and virus-like particles (VLPs); and
(vii) optionally: further purifying the immunogenic composition and thus obtaining, separated from each other, recombinant virus-like particles (VLPs) and/or infectious virus particles.

15. A method of preventing or treating a Norovirus virus disease in a subject, comprising administering the immunogenic composition according to claim 1 to the subject.

16. The immunogenic composition according to claim 2, wherein the Norovirus VP1 sequence and/or the Norovirus VP2 sequence are independently derived from a sequence from a Norovirus Hu/GII.4/Sydney/NSW0514/2012/AU, a Norovirus Hu/GII.4/Orange/NSW001P/2008/AU, a Norovirus Hu/GII.4/NIHIC17.5/2012/USA, a Norovirus Hu/GII.4/GII4-NG1242/2011/JP, or a Norovirus Hu/GII.4/Hiroshima4/2012/JP.

17. The immunogenic composition according to claim 3, wherein the vector backbone is from a recombinant live-attenuated measles virus strain.

18. The immunogenic composition according to claim 8, wherein the at least two different Norovirus genogroups are a GII genogroup and a GI genogroup.

19. The immunogenic composition according to claim 1, wherein the at least one nucleic acid sequence encoding the at least one Norovirus VP1 sequence and/or the at least one nucleic acid sequence encoding at least one Norovirus VP2 sequence are selected from different Norovirus GII genotypes.

20. The immunogenic composition according to claim 19, wherein the different Norovirus GII genotypes are (a) a GII.4 genotype and (b) a GII.17 genotype or a GII.2 genotype.
